SEQUENCE LISTING (2 sequences)

SEQ ID NO: 1 - DNA, 15 bp, Artificial Sequence
  stem-loop secondary structure
acucagcgauaaagu

SEQ ID NO: 2 - DNA, 13 bp, Artificial Sequence
  staple secondary structure
aacggaacacgaa

(19) United States
(12) Patent Application Publication
(10) Pub. No.: US 2021/0230680 A1
(43) Pub. Date: Jul. 29, 2021

(54) Modified Nucleobases with Uniform H-Bonding Interactions, Homo- and Hetero-Basepair Bias, and Mismatch Discrimination

(71) Applicant: Carnegie Mellon University, Pittsburgh, PA (US)

(72) Inventors: Danith H. Ly, Pittsburgh, PA (US); Shivaji A. Thadke, Pittsburgh, PA (US); Ashif Y. Shaikh, Maharashtra (IN); Wei-Che Hsieh, New Taipei City (TW); Ali Nakhi, Falcon Heights, MN (US); J. Dinithi Perera, Pittsburgh, PA (US)

(21) Appl. No.: 16/972,873

(22) PCT Filed: Jun. 7, 2019
(86) PCT No.: PCT/US19/36017

§ 371 Date: Dec. 7, 2020

Related U.S. Application Data
(60) Provisional application No. 62/763,299, filed on Jun. 8, 2018.

(51) Int. Cl. C12Q 1/6832; C07K 14/00; C07H 21/00

(52) U.S. Cl. CPC C12Q 1/6832; C07H 21/00; C07K 14/003

(57) ABSTRACT

Described herein are divalent nucleobases that each binds two nucleic acid strands, matched or mismatched when incorporated into a nucleic acid or nucleic acid analog backbone, such as in a γ-peptide nucleic acid (γPNA). Also provided are genetic recognition reagents comprising one or more of the divalent nucleobases and a nucleic acid or nucleic acid analog backbone, such as a γPNA backbone. Uses for the divalent nucleobases and monomers and genetic recognition reagents containing the divalent nucleobases also are provided.

### CROSS REFERENCE TO RELATED APPLICATIONS

This application is the United States national phase of International Application No. PCT/US2019/036017 filed Jun. 7, 2019, and claims the benefit of U.S. Provisional Patent Application No. 62/763,299 filed Jun. 8, 2018, the disclosures of which are hereby incorporated by reference in their entirety.

The Sequence Listing associated with this application is filed in electronic format via EFS-Web and is hereby incorporated by reference into the specification in its entirety. The name of the text filed containing the Sequence Listing is 6526_2006675_ST25.txt. The size of the text file is 921 bytes, and the text filed was created Nov. 12, 2020.

### BACKGROUND

Described herein are nucleobases, polymer monomers comprising the nucleobases and nucleic acids and analogs thereof comprising the nucleobases. Also described herein are methods of use of the nucleobases, polymer monomers comprising the nucleobases and nucleic acids and analogs thereof comprising the nucleobases.

For most organisms, genetic information is encoded in double-stranded DNA in the form of Watson-Crick base-pairing—in which adenine (A) pairs with thymine (T) and cytosine (C) with guanine (G). Depending on which set of this genetic information is decoded through transcription and translation, the developmental program and physiological status will be determined. Development of molecules that can be tailor-designed to bind sequence-specifically to any part of this genetic biopolymer (DNA or RNA), thereby enabling the control of the flow of genetic information and assessment and manipulation of the genome's structures and functions, is important for biological and biomedical research in the effort to unravel the molecular basis of life, including molecular tools for basic research in biology. This effort is also important for medicinal and therapeutic applications for the treatment and detection of genetic diseases.

Oligonucleotides are versatile in their use as molecular tools for basic research in biology, as well as molecular reagents for therapeutic and diagnostics applications, e.g., in the treatment and detection of genetic diseases. Generally, oligonucleotide molecules are short pieces (10-30 nucleotides in length) of single-stranded DNA or RNA, or derivatives thereof. They may include sugar phosphodiester backbones, or other backbones, that are connected to the adenine (A), cytosine (C), guanine (G), and thymine (T) or uridine (U) nucleobases. They are designed to bind to the DNA or RNA targets through Watson-Crick base-pairing in which A pairs with T (or U) and C with G. Oligonucleotide molecules have been employed in a broad range of applications, including, for example, interrogation of nucleic acid sequence information, manipulation of RNA structure, and regulation of gene expression. The success of many of these applications rely on the ability of the oligomers to bind to DNA or RNA targets in a tight and sequence-specific manner. Additional requirements for intracellular and in vivo gene targeting include enzymatic stability and cell permeability.

### SUMMARY

A genetic recognition reagent is provided, comprising a plurality of nucleobase moieties attached to a nucleic acid or nucleic acid analog backbone, in which at least one nucleobase moiety is:

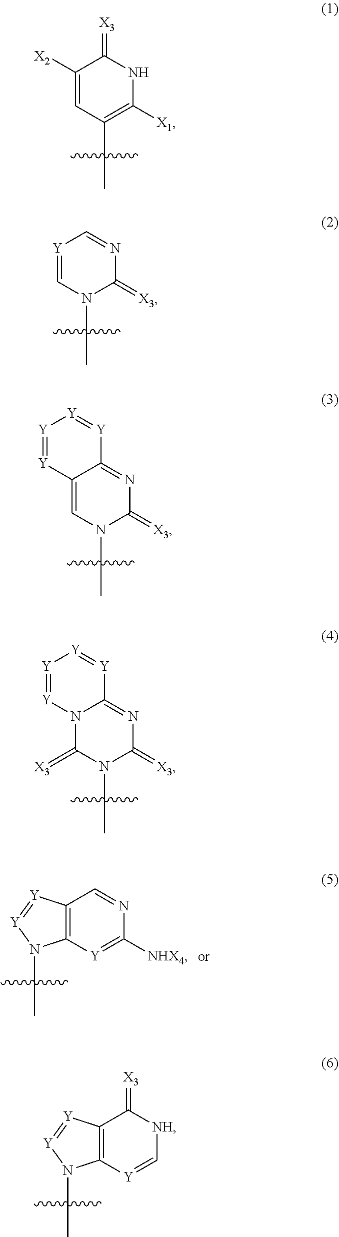

wherein, X₁ is ═O (═ referring to a double bond), ═S, ═Se, or CH₃; X₂ is H, CH₃, CN, NC, N₃, C(O)OH, or C(O)NH₂; X₃ is O or S; X₄ is H, C(O)CH₃, or C(O)OCH₃; and Y is N or CH, wherein in (1), when X₁ and X₃ are O, X₂ is not H or methyl.

A compound also is provided, comprising a nucleic acid backbone monomer or nucleic acid analog backbone monomer linked to a nucleobase moiety having the structure:

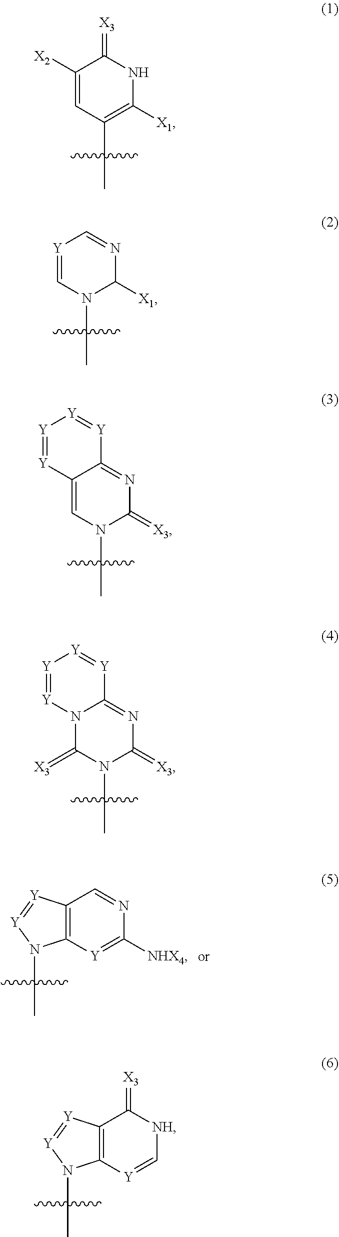

wherein, X₁ is ═O, ═S, ═Se, or CH₃; X₂ is H, CH₃, CN, NC, N₃, C(O)OH, or C(O)NH₂; X₃ is O or S; X₄ is H, C(O)CH₃, or C(O)OCH₃; and Y is N or CH, wherein in (I), when X₁ and X₃ are O, X₂ is not H or methyl.

### BRIEF DESCRIPTION OF THE DRAWINGS

FIG. 1 illustrates the hydrogen-bonding interactions of (A) modified vs. modified nucleobases (homoduplex), (B) natural vs. natural, (C) modified vs. natural (heteroduplex).

FIG. 2 is schematic diagrams showing a distinct advantage that the newly designed oligonucleotide molecules provide in being able to selectively target RNA secondary structure. (A) Despite the (a′/a) sequence complementarity, a PNA oligomer (chiral or achiral) containing modified (u, c, a, and g) nucleobases cannot adopt a hairpin structure and is able to hybridize to its complementary stem-loop RNA target. (B) Tight-binding oligonucleotide molecule such as LNA or yPNA is able to invade the stem-loop structure, but its application in therapeutics and diagnostics poses considerable risks due to the nonspecific binding. (C) Moderate avidity oligonucleotides, a category in which most oligonucleotide molecules fall under, are unable to open the stem-loop structure due to the lack of binding free energy, or as the result of the formation of a kinetic (hairpin) trap.

FIG. 3 schematically depicts examples of RNA (A) secondary (SEQ ID NO. 1 and SEQ ID NO. 2) and (B) tertiary structures that oligonucleotide molecules described herein are able to selectively target that would otherwise be difficult to accomplish with existing nucleic acid systems.

FIG. 4 provides structures of exemplary nucleobases.

FIG. 5 depicts exemplary nucleic acid analog residues for nucleic acid analogs, including: phosphorothioate DNA (PS DNA), α, β-constrained nucleic acid (α,β-CNA), 2′-methoxyl RNA, 2′-fluoro RNA, phosphorodiamidate morpholino oligomer (PMO), locked nucleic acid (LNA), 2′,4′-constrained ethyl nucleic acid ((S)-cEt), 2′,4′ bridged nucleic acid NC (N—H) (BNA-NC(N—H)), 2′,4′ bridged nucleic acid NC (N-methyl) (BNA-NC(N-Me)), ((S)-5′-C-methyl DNA (RNA)), and 5′-E-vinylphosphonate nucleic acid (E-VP), wherein R is H, OH, F, OMe, or O(CH₂)₂OMe.

FIG. 6 provides exemplary synthetic schemes for selected nucleobases.

FIG. 7 provides an exemplary synthetic scheme for selected cell-permeable γPNA monomers.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and can be synonymous with “including”, “containing”, or “characterized by”. As used herein, embodiments “comprising” one or more stated elements or steps also include, but are not limited to embodiments “consisting essentially of” and “consisting of” these stated elements or steps.

The term “polymer composition” is a composition comprising one or more polymers. As a class, “polymers” include, without limitation, homopolymers, heteropolymers, co-polymers, block polymers, block co-polymers and can be both natural and synthetic. Homopolymers contain one type of building block, or monomer, whereas co-polymers contain more than one type of monomer. An “oligomer” is a polymer that comprises a small number of monomers, such as, for example, from 3 to 100 monomer residues. As such, the term “polymer” includes oligomers. The terms “nucleic acid” and “nucleic acid analog” includes nucleic acid and nucleic acid polymers and oligomers.

A polymer “comprises” or is “derived from” a stated monomer if that monomer is incorporated into the polymer. Thus, the incorporated monomer that the polymer comprises is not the same as the monomer prior to incorporation into a polymer, in that at the very least, certain linking groups are incorporated into the polymer backbone or certain groups are removed in the polymerization process. A polymer is said to comprise a specific type of linkage if that linkage is present in the polymer. An incorporated monomer is a “residue”. A typical monomer for a nucleic acid or nucleic acid analog is referred to as a nucleotide or a nucleotide residue when incorporated into a polymer.

A “moiety” (pl. “moieties”) is a part of a chemical compound, and comprises groups, such as functional groups. As such, a nucleobase moiety is a nucleobase that is modified by attachment to another compound moiety, such as a polymer monomer, e.g. the nucleic acid or nucleic acid analog monomers described herein, or a polymer, such as a nucleic acid or nucleic acid analog as described herein.

“Alkyl” refers to straight, branched chain, or cyclic hydrocarbon groups including from 1 to about 20 carbon atoms, for example and without limitation C₁₋₃, C₁₋₆, C₁₋₁₀ groups, for example and without limitation, straight, branched chain alkyl groups such as methyl, ethyl, propyl, butyl, pentyl, hexyl, heptyl, octyl, nonyl, decyl, undecyl, dodecyl, and the like. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of straight alkyl groups include methyl, ethyl, propyl, butyl, pentyl, hexyl, heptyl, octyl, nonyl, and decyl. Branched alkyl groups comprises any straight alkyl group substituted with any number of alkyl groups. Non-limiting examples of branched alkyl groups include isopropyl, isobutyl, sec-butyl, and t-butyl. Non-limiting examples of cyclic alkyl groups include cyclopropyl, cyclobutyl, cyclopentyl, cyclohexyl, cycloheptlyl, and cyclooctyl groups. Cyclic alkyl groups also comprise fused-, bridged-, and spiro-bicycles and higher fused-, bridged-, and spiro-systems. A cyclic alkyl group can be substituted with any number of straight, branched, or cyclic alkyl groups.

“Substituted alkyl” refers to alkyl substituted at 1 or more, (e.g., 1, 2, 3, 4, 5, or 6) positions, which substituents are attached at any available atom to produce a stable compound, with substitution as described herein. “Optionally substituted alkyl” refers to alkyl or substituted alkyl. “Halogen,” “halide,” and “halo” refers to —F, —Cl, —Br, and/or —I. “Alkylene” and “substituted alkylene” refer to divalent alkyl and divalent substituted alkyl, respectively, including, without limitation, ethylene (—CH₂—CH₂—). “Optionally substituted alkylene” refers to alkylene or substituted alkylene.

“Alkene or alkenyl” refers to straight, branched chain, or cyclic hydrocarbyl groups including from 2 to about 20 carbon atoms, such as, without limitation C₂₋₃, C₂₋₆, C₂₋₁₀ groups having one or more (e.g., 1, 2, 3, 4, or 5) carbon-to-carbon double bonds. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. A halo-alkenyl group can be any alkenyl group substituted with any number of halogen atoms.

“Substituted alkene” refers to alkene substituted at 1 or more (e.g., 1, 2, 3, 4, or 5 positions) which substituents are attached at any available atom to produce a stable compound, with substitution as described herein. “Optionally substituted alkene” refers to alkene or substituted alkene. Likewise, “alkenylene” refers to divalent alkene. Examples of alkenylene include without limitation, ethenylene (—CH═CH—) and all stereoisomeric and conformational isomeric forms thereof. “Substituted alkenylene” refers to divalent substituted alkene. “Optionally substituted alkenylene” refers to alkenylene or substituted alkenylene.

Alkyne or “alkynyl” refers to a straight, branched chain, or cyclic unsaturated hydrocarbon having the indicated number of carbon atoms and at least one triple bond. The triple bond of an alkyne or alkynyl group can be internal or terminal. Examples of a (C₂-C₈)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein below. An alkyne or alkynyl group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. A halo-alkynyl group can be any alkynyl group substituted with any number of halogen atoms.

The term “alkynylene” refers to divalent alkyne. Examples of alkynylene include without limitation, ethynylene, propynylene. “Substituted alkynylene” refers to divalent substituted alkyne.

The term “alkoxy” refers to an —O-alkyl group having the indicated number of carbon atoms. An ether or an ether group comprises an alkoxy group. For example, a (C₁-C₆)alkoxy group includes —O-methyl (methoxy), —O-ethyl (ethoxy), —O— propyl (propoxy), —O-isopropyl (isopropoxy), —O-butyl (butoxy), —O-sec-butyl (sec-butoxy), —O-tert-butyl (tert-butoxy), —O-pentyl (pentoxy), —O-isopentyl (isopentoxy), —O-neopentyl (neopentoxy), —O-hexyl (hexyloxy), —O-isohexyl (isohexyloxy), and —O— neohexyl (neohexyloxy). “Hydroxyalkyl” refers to a (C₁-C₁₀)alkyl group wherein one or more of the alkyl group's hydrogen atoms is replaced with an —OH group. Examples of hydroxyalkyl groups include, but are not limited to, —CH₂OH, —CH₂CH₂OH, —CH₂CH₂CH₂OH, —CH₂CH₂CH₂CH₂OH, —CH₂CH₂CH₂CH₂CH₂OH, —CH₂CH₂CH₂CH₂CH₂CH₂OH, and branched versions thereof. The term “ether” or “oxygen ether” refers to (C₁-C₁₀)alkyl group wherein one or more of the alkyl group's carbon atoms is replaced with an —O— group. The term ether includes —CH₂—(OCH₂—CH₂)_{q}OP₁ compounds where P₁ is a protecting group, —H, or a (C₁-C₁₀)alkyl. Exemplary ethers include polyethylene glycol, diethylether, methylhexyl ether and the like.

The term “thioether” refers to (C₁-C₁₀)alkyl group wherein one or more of the alkyl group's carbon atoms is replaced with an —S— group. The term thioether includes —CH₂—(SCH₂—CH₂)_{q}—SP₁ compounds where P₁ is a protecting group, —H, or a (C₁-C₁₀)alkyl. Exemplary thioethers include dimethylthioether or ethylmethyl thioether.

Protecting groups (e.g., for protecting amines during synthesis of compounds described herein) are known in the art and include, without limitation: 9-fluorenylmethyloxy carbonyl (Fmoc), t-butyloxycarbonyl (Boc), benzhydryloxycarbonyl (Bhoc), benzyloxycarbonyl (Cbz), O-nitroveratryloxycarbonyl (Nvoc), benzyl (Bn), allyloxycarbonyl (alloc), trityl (Trt), dimethoxytrityl (DMT), I-(4,4-dimethyl-2,6-dioxacyclohexylidene)ethyl (Dde), diathiasuccinoyl (Dts), benzothiazole-2-sulfonyl (Bts) and monomethoxytrityl (MMT) groups.

“Aryl,” alone or in combination refers to an aromatic monocyclic or bicyclic ring system such as phenyl or naphthyl. “Aryl” also includes aromatic ring systems that are optionally fused with a cycloalkyl ring. A “substituted aryl” is an aryl that is independently substituted with one or more substituents attached at any available atom to produce a stable compound, wherein the substituents are as described herein. The substituents can be, for example, hydrocarbyl groups, alkyl groups, alkoxy groups, and halogen atoms. “Optionally substituted aryl” refers to aryl or substituted aryl. An aryloxy group can be, for example, an oxygen atom substituted with any aryl group, such as phenoxy. An arylalkoxy group can be, for example, an oxygen atom substituted with any aralkyl group, such as benzyloxy.

“Arylene” denotes divalent aryl, and “substituted arylene” refers to divalent substituted aryl. “Optionally substituted arylene” refers to arylene or substituted arylene.

“Heteroatom” refers to N, O, P and S. Compounds that contain N or S atoms can be optionally oxidized to the corresponding N-oxide, sulfoxide or sulfone compounds. “Hetero-substituted” refers to an organic compound in any embodiment described herein in which one or more carbon atoms are substituted with N, O, P or S.

“Cycloalkyl” refer to monocyclic, bicyclic, tricyclic, or polycyclic, 3- to 14-membered ring systems, which are either saturated, unsaturated or aromatic. The cycloalkyl group may be attached via any atom. Cycloalkyl also contemplates fused rings wherein the cycloalkyl is fused to an aryl or heteroaryl ring. Representative examples of cycloalkyl include, but are not limited to cyclopropyl, cyclobutyl, cyclopentyl, and cyclohexyl. A cycloalkyl group can be unsubstituted or optionally substituted with one or more substituents as described herein below. “Cycloalkylene” refers to divalent cycloalkyl. The term “optionally substituted cycloalkylene” refers to cycloalkylene that is substituted with 1, 2 or 3 substituents, attached at any available atom to produce a stable compound, wherein the substituents are as described herein.

“Carboxyl” or “carboxylic” refers to group having the indicated number of carbon atoms and terminating in a —C(O)OH group, thus having the structure —R—C(O)OH, where R is a divalent organic group that includes linear, branched, or cyclic hydrocarbons. Non-limiting examples of these include: C₁₋₈ carboxylic groups, such as ethanoic, propanoic, 2-methylpropanoic, butanoic, 2,2-dimethylpropanoic, pentanoic, etc.

“(C₃-C₈)aryl-(C₁-C₆)alkylene” refers to a divalent alkylene wherein one or more hydrogen atoms in the C₁-C₆ alkylene group is replaced by a (C₃-C₈)aryl group. Examples of (C₃-C₈)aryl-(C₁-C₆)alkylene groups include without limitation 1-phenylbutylene, phenyl-2-butylene, 1-phenyl-2-methylpropylene, phenylmethylene, phenylpropylene, and naphthylethylene. The term “(C₃-C₈)cycloalkyl-(C₁-C₆)alkylene” refers to a divalent alkylene wherein one or more hydrogen atoms in the C₁-C₆ alkylene group is replaced by a (C₃-C₈)cycloalkyl group. Examples of (C₃-C₈)cycloalkyl-(C₁-C₆)alkylene groups include without limitation 1-cycloproylbutylene, cycloproyl-2-butylene, cyclopentyl-1-phenyl-2-methylpropylene, cyclobutylmethylene and cyclohexylpropylene.

A moiety, such as a nucleobase moiety, functional group, guanidine-containing group, or PEG-containing group, in a larger molecule, such as a nucleic acid or nucleic acid polymer chain, e.g., as described herein, is “linked” to the remainder of the molecule, meaning it is covalently-attached either directly, or through an inert linking moiety, to the remainder of the molecule. By “inert,” it is meant, that the linker does not substantially affect the function of the nucleic acid or nucleic acid analog for its intended use. Non-limiting examples of inert linkers include linear, branched, and/or cyclic hydrocarbyl or substituted hydrocarbyl moieties, for example having less than 15, 10, or 6 carbon atoms, such as an alkylene moiety, e.g., a methylene, ethylene, propylene, or butylene group, or an aryl group, and optionally containing a heteroatom, such an S (e.g., thioether), N (tertiary amine), or O (e.g., ether) atom, and/or a linking bond, such as, for example and without limitation, an ester, amide, carbamate, or carbonate linkage. A linker may serve as a spacer for physically or spatially separating two constituents of a molecule.

Provided herein are nucleic acids and analogs thereof, collectively “genetic recognition reagents”, that bind specifically to a nucleic acid strand, e.g., under physiological conditions, e.g., in normal saline (0.9% wt. NaCl), or another isotonic solution such as Tris-buffered saline or PBS, at 37° C. The genetic recognition reagent comprises a plurality of nucleobase moieties, each attached to a nucleic acid or nucleic acid analog backbone monomer residue, e.g., in the case of DNA or RNA, forming a nucleoside or, with the phosphate group, a nucleotide), and forming a part of the larger genetic recognition reagent comprising at least two nucleic acid or nucleic acid monomer residues, and therefore at least two nucleobase moieties.

In one aspect, all nucleobases of the genetic recognition reagents are modified nucleobases as described herein. In another embodiment, the genetic recognition reagents described herein comprise at least one modified nucleobase as described herein, with other nucleobases being natural nucleobases (e.g. adenine, guanine, cytosine, thymine, or uracil), or different from those modified bases.

Thus in one aspect, modified nucleobases are provided. Those nucleobases can be incorporated into a nucleic acid or nucleic acid analog monomer (e.g., nucleotide), which can then be incorporated into an oligomer or polymer of monomers with a desired sequence of nucleobases. Structures of the modified nucleobases are provided below, and include:

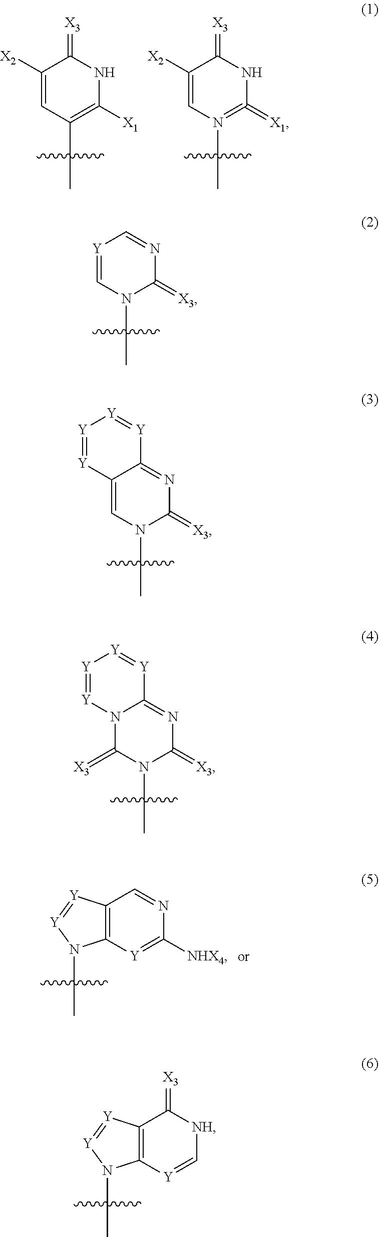

wherein, wherein,

X₁ is ═O (═ referring to a double bond), ═S, ═Se, or CH₃;

X₂ is H, CH₃, CN, NC, N₃, C(O)OH, or C(O)NH₂;

X₃ is O or S;

X₄ is H, C(O)CH₃, or C(O)OCH₃; and

Y is N or CH,

wherein in (1), when X₁ and X₃ are O, X₂ is not H or methyl. Non-limiting examples of such nucleobases, forming a complete set of nucleobases able to bind A, T, G, and C of natural DNA or RNA, include:

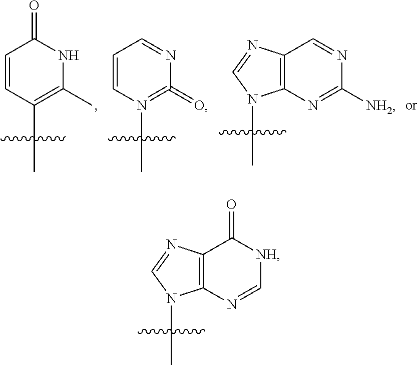

Additional examples of such nucleobases include the fluorescent bases:

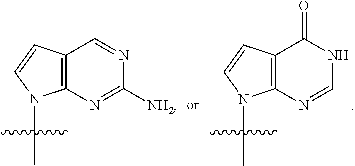 [SMILES: *n1ccc2cnc(N)nc21 ; *n1ccc2c(=O)[nH]cnc21]

Any, some, or all of the preceding nucleobases may be incorporated into nucleotide monomers and a genetic recognition reagent.

In one aspect, the genetic recognition reagents described herein are capable of self-assembly on a nucleic acid template comprising a target sequence of the genetic recognition reagents. For example, a first genetic recognition reagent can hybridize to a first portion of a nucleic acid template. A second self-assembling genetic recognition reagent can hybridize to a second portion, adjacent to the first portion, of the nucleic acid template. The first genetic recognition reagent and the second genetic recognition reagent can either covalently-link or non-covalently-link together using various functional end groups to form a contiguous structure. The genetic recognition reagents can comprise a first moiety linked by a linker to the first end of the nucleic acid or nucleic acid analog backbone and a second moiety linked by a linker to the second end of the nucleic acid or nucleic acid analog backbone. The second moiety can be the same as the first moiety. The second moiety can be different than the first moiety. In one example, International Patent Application No. PCT/US18/67096, incorporated herein by reference, discloses self-assembling genetic recognition reagents that comprise aryl groups at their ends that pi-stack to form strong non-covalent linkages between unit genetic recognition reagents when aligned contiguously on a target sequence, for example an expanded repeat, such as TTC, TTCTTC, TCT, TCTTCT, CTT, CTTCTT, CCG, CCGCCG, CGC, CGCCGC, GCC, GCCGCC, CGG, CGGCGG, GCG, GCGGCG, GGC, GGCGGC, CTG, CTGCTG, TGC, TGCTGC, GCT, GCTGCT, CAG, CAGCAG, AGC, AGCAGC, GCA, GCAGCA, CAGG, CAGGCAGG, AGGC, AGGCAGGC, GGCA, GGCAGGCA, GCAG, GCAGGCAG, AGAAT, GAATA, AATAG, ATAGA, TAGAA, GGCGGC, GCGGCG, CGGCGG, GGCGGC, CCGGCC, and CGGCGG. In another example of self-assembling genetic recognition reagents, International Patent Application Publication No. WO 2014/169216, incorporated herein by reference in its entirety, describes genetic recognition reagents that self-assemble and covalently-link in a reducing environment by virtue of terminal thioester and sulfhydryl groups. Covalent linkages form between unit self-assembling genetic recognition reagents when aligned contiguously on a target sequence, for example on a nucleic acid comprising an expanded repeat, e.g., as described above. Other end-groups, such as affinity binding partners, or reactive groups, can be attached to the ends of the genetic recognition reagents described herein to render them capable of self-assembly on a complementary nucleic acid template.

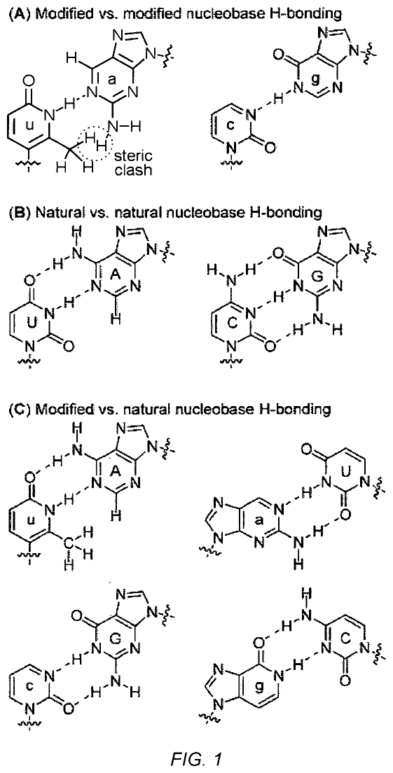

In one embodiment, provided herein is a compound comprising a nucleobase and a nucleic acid or nucleic acid analog backbone monomer. In the context of the present disclosure, a “nucleotide” refers to a compound or residue of a genetic recognition reagent comprising at least one nucleobase and a backbone element, which in a nucleic acid, such as RNA or DNA is ribose or deoxyribose. Nucleotide monomers also comprise reactive groups that permit polymerization under specific conditions. In native DNA and RNA, those reactive groups are the 5′ phosphate and 3′ hydroxyl groups. For chemical synthesis of nucleic acids and analogs thereof, the bases and backbone monomers may contain modified groups, such as blocked amines, as are known in the art. A “nucleotide residue” refers to a single nucleotide that is incorporated into an oligonucleotide or polynucleotide. Likewise, a “nucleobase residue” refers to a nucleobase incorporated into a nucleotide or a nucleic acid or analog thereof. A “genetic recognition reagent” refers generically to a nucleic acid or a nucleic acid analog that comprises a sequence of nucleobases that is able to hybridize to a complementary nucleic acid sequence on a nucleic acid by cooperative base pairing (e.g., Watson-Crick base pairing or Watson-Crick-like base pairing) (see, FIG. 1). Intra-molecular base pairing does not occur between the modified bases described herein, where there is either steric clash or insufficient hydrogen bonding between bases that would normally base-pair in natural nucleic acid (FIG. 1, panel (A) (“FIG. 1 (A)”)). As can be seen in FIG. 1 (B), normal base-pairing is asymmetrical, with two hydrogen bonds forming between A and T/U nucleobases, and three hydrogen bonds forming between G and C nucleobases. FIG. 1 (C) depicts a significant benefit of use of the modified nucleobases described herein, in that binding is symmetrical, with two hydrogen bonds forming between all combinations of the modified nucleobases described herein and their natural base-pairing partner. Not only does the near equal binding “weight” distribution of base-pairs greatly simplify the probe design, with the binding strength of the probe heavily reliance on length rather than on length and sequence composition, it confers greater sequence discrimination against base-pair mismatches than that could be achieved with natural nucleobases.

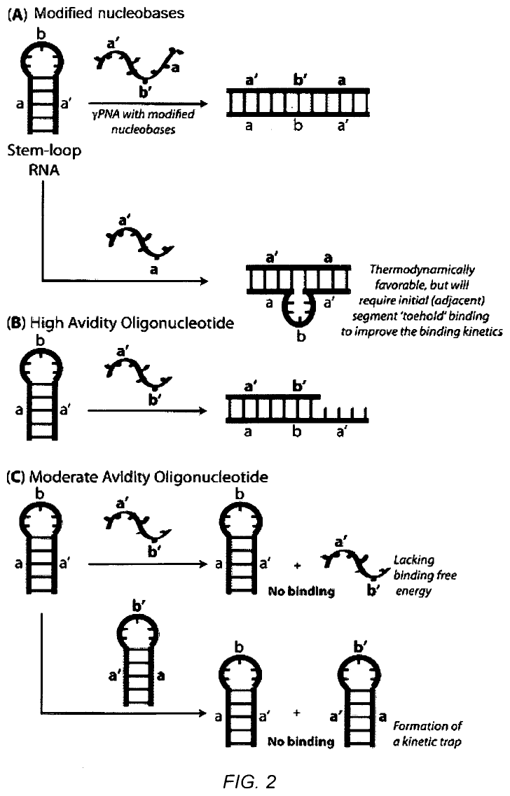

FIG. 2 depicts a benefit of the modified nucleobases described herein. FIG. 2 (A) shows that despite the (a′/a) sequence complementarity, a PNA oligomer (chiral or achiral) containing modified (u, c, a, and g) nucleobases cannot adopt a hairpin structure, but is able to hybridize to its complementary stem-loop RNA target. FIG. 2 (B) shows that a tight-binding oligonucleotide molecule such as Locked Nucleic Acid (LNA) or PNA (e.g., γPNA) is able to invade the stem-loop structure, but its application in therapeutics and diagnostics poses considerable risks due to nonspecific binding. FIG. 2 (C) shows that moderate avidity oligonucleotides, a category in which most oligonucleotide molecules fall under, are unable to open the stem-loop structure due to the lack of binding free energy, or as the result of the formation of a kinetic (hairpin) trap.

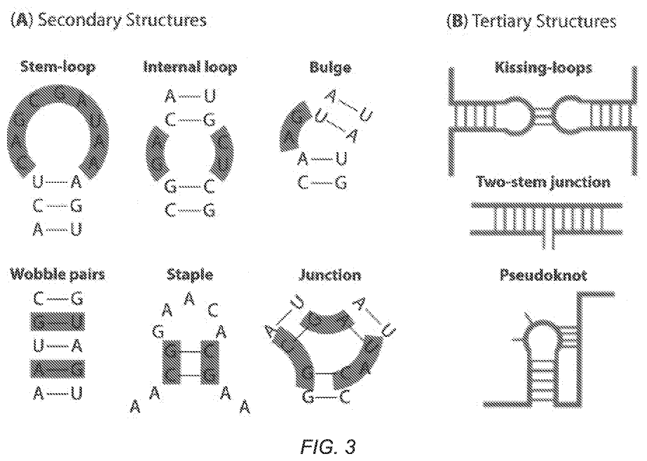

Examples of RNA secondary (SEQ ID NO. 1 and SEQ ID NO. 2) and tertiary structures that could be selectively targeted are shown in FIG. 3. Potential therapeutic and diagnostic targets comprise RNA, both coding and noncoding, and DNA.

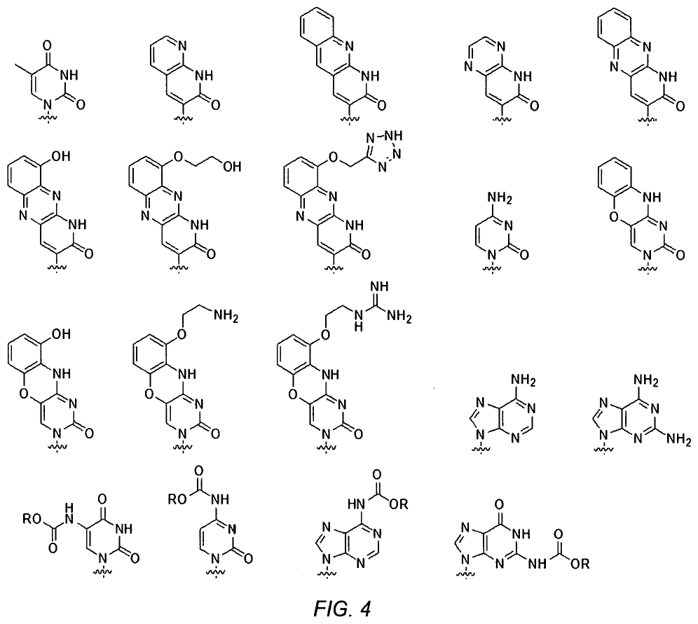

In aspects, provided herein are modified nucleobases. Nucleobases are recognition moieties that, e.g., bind specifically to one or more of adenine, guanine, thymine, cytosine, and uracil, e.g., by Watson-Crick or Watson-Crick-like base pairing by hydrogen bonding. A “nucleobase” includes primary (natural) nucleobases: adenine, guanine, thymine, cytosine, and uracil, as well as modified purine and pyrimidine bases, such as, without limitation, hypoxanthine, xanthene, 7-methylguanine, 5, 6, dihydrouracil, 5-methylcytosine, and 5-hydroxymethylcytosine. FIG. 4 also depicts non-limiting examples of nucleobases, including monovalent nucleobases (e.g., adenine, cytosine, guanine, thymine or uracil, which bind to one strand of nucleic acid or nucleic acid analogs), and “clamp” nucleobases, such as a “G-clamp,” which binds complementary nucleobases with enhanced strength. Additional purine, purine-like, pyrimidine and pyrimidine-like nucleobases are known in the art, for example as disclosed in U.S. Pat. Nos. 8,053,212, 8,389,703, and 8,653,254. Divalent nucleobases are described in further detail in United States Patent Application Publication No. 2016/0083434 A1 and International Patent Application Publication No. WO/2018/058091, all of which are incorporated herein by reference, and bind two nucleobases instead of one, and therefore can form complex trimeric structures with matched or mismatched nucleic acids.

In one example the backbone monomer is a ribose mono-, di-, or tri-phosphate or a deoxyribose mono-, di-, or tri-phosphate, such as a 5′ monophosphate, diphosphate, or triphosphate of ribose or deoxyribose. The backbone monomer includes both the structural “residue” component, such as the ribose in RNA, and any active groups that are modified in linking monomers together, such as the 5′ triphosphate and 3′ hydroxyl groups of a ribonucleotide, which are modified when polymerized into RNA to leave a phosphodiester linkage. Likewise for PNA, the C-terminal carboxyl and N-terminal amine active groups of the N-(2-aminoethyl)glycine backbone monomer are condensed during polymerization to leave a peptide (amide) bond. In another aspect, the active groups are phosphoramidite groups useful for phosphoramidite oligomer synthesis, as is broadly-known in the arts. The nucleotide monomer also optionally comprises one or more protecting groups as are known in the art, such as 4,4′-dimethoxytrityl (DMT), and as described herein. A number of additional methods of preparing synthetic genetic recognition reagents are known, and depend on the backbone structure and particular chemistry of the base addition process. Determination of which active groups to utilize in joining nucleotide monomers and which groups to protect in the bases, and the required steps in preparation of oligomers is well within the abilities of those of ordinary skill in the chemical arts and in the particular field of nucleic acid and nucleic acid analog oligomer synthesis.

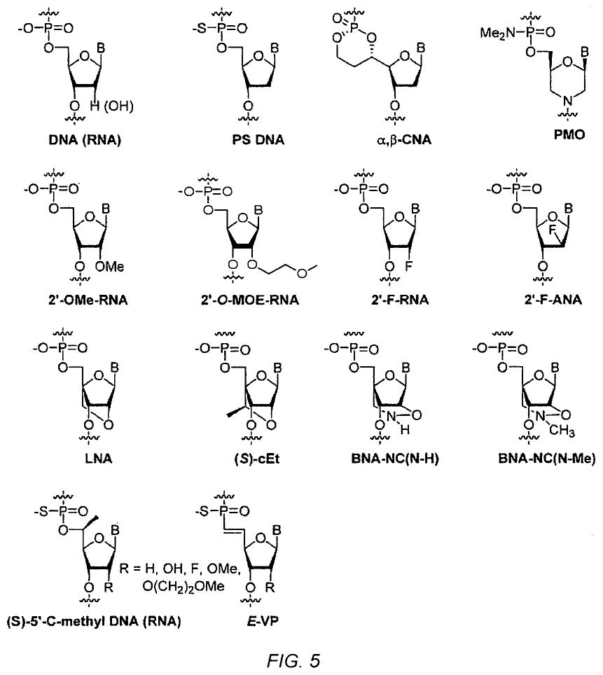

As used herein, the term “nucleic acid” refers to deoxyribonucleic acids (DNA) and ribonucleic acids (RNA). Nucleic acid analogs include, for example and without limitation (see, e.g., FIG. 5): phosphorothioate DNA (PS DNA), α, β-constrained nucleic acid (α,β-CNA), 2′-methoxyl RNA, 2′-fluoro RNA, phosphorodiamidate morpholino oligomer (PMO), locked nucleic acid (LNA), 2′,4′-constrained ethyl nucleic acid ((S)-cEt), 2′,4′ bridged nucleic acid NC (N—H) (BNA-NC(N—H)), 2′,4′ bridged nucleic acid NC (N-methyl) (BNA-NC(N-Me)), ((S)-5′-C-methyl DNA (RNA)), and 5′-E-vinylphosphonate nucleic acid (E-VP), wherein R is H, OH, F, OMe, or O(CH₂)₂OMe and combinations thereof including, optionally ribonucleotide or deoxyribonucleotide residue(s). An “oligonucleotide” is a short, single-stranded genetic recognition reagent. An oligonucleotide may be referred to by the length (i.e. number of nucleotides or nucleobases) of the strand, through the nomenclature “-mer”. For example, an oligonucleotide of 22 nucleotides would be referred to as a 22-mer.

A “peptide nucleic acid” refers to a DNA or RNA analog or mimic in which the sugar phosphodiester backbone of the DNA or RNA is replaced by a N-(2-aminoethyl)glycine unit. In one embodiment, the peptide nucleic acid has the structure:

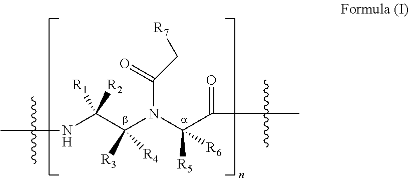

where n is 1 or greater, R₁, R₂, R₃, R₄, R₅, and R₆ are, independently: H; CH₃, CH₂OH, CH(CH₃)OH, CH₂SH, CH(CH₃)CH₃, CH₂CH(CH₃)CH₃, CH(CH₃)CH₂CH₃, CH₂CH₂SCH₃, CH₂CH₃, CH₂—C₆H₅, CH₂—C₆H₄OH, 1H-indol-3-yl methyl, CH₂C(O)OH, CH₂CH₂C(O)OH, CH₂C(O)NH₂, CH₂CH₂C(O)NH₂, 1 H-imidazol-4-yl methyl, CH₂CH₂CH₂CH₂NH₂, or CH₂CH₂CH₂NHC(NH)NH₂; linear or branched (C₃-C₈)alkyl, (C₂-C₈)alkenyl, (C₂-C₈)alkynyl, (C₃-C₈)aryl, (C₃-C₈)cycloalkyl, (C₃-C₈)aryl(C₁-C₆)alkylene, (C₃-C₈)cycloalkyl(C₁-C₆)alkylene, a guanidine-containing group, CH₂—(OCH₂—CH₂)ₙ—OH, CH₂—(OCH₂—CH₂)ₙ—NH₂, CH₂—(OCH₂—CH₂)ₙ—SH, CH₂—(OCH₂—CH₂)ₙ—NHC(NH)NH₂, CH₂—(OCH₂—CH₂)ₙ-morpholine, CH₂—(OCH₂—CH₂)ₙ-piperazine,

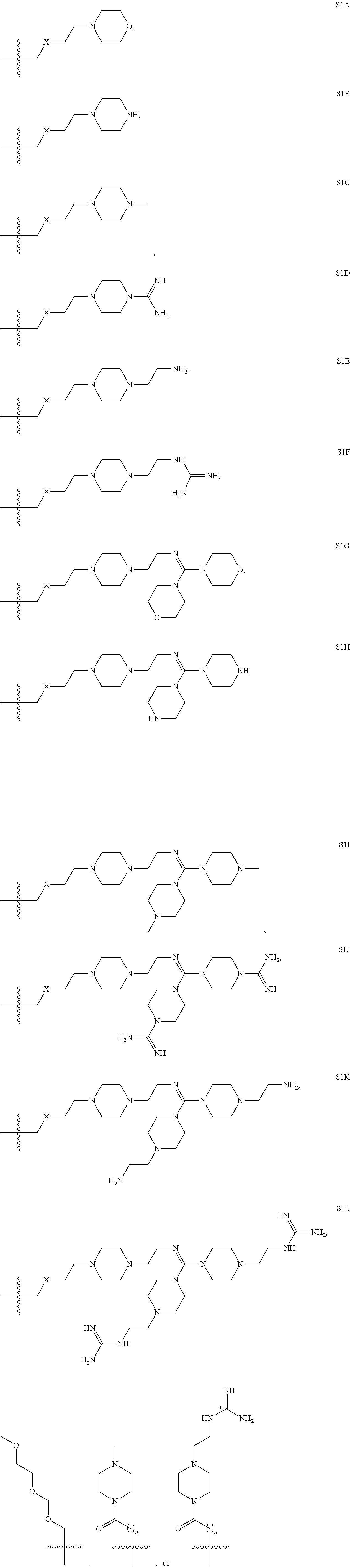

wherein X is a linker, such as a linear linker, R₁ and R₂ together form a 1,3-propylene linkage, R₃ and R₄ together form a 1,3-propylene linkage, or R₅ and R₆ together form a 1,3-propylene linkage, and each instance of R₇ is, independently, a nucleobase, forming a nucleobase sequence where n is two or greater.

A linker is a moiety in a compound that covalently connects one moiety to another. It imparts no substantial negative effect on the activity of the overall compound, e.g., in context of the present invention, the ability of a genetic recognition reagent to operate in its intended use. Aside from serving to covalently-link two moieties, a linker may have a beneficial effect, such as in the physical separation of moieties to which it is attached, e.g., to optimize spacing to avoid steric effects. A linker also may serve some additional function, such as altering the hydrophobicity/hydrophilicity of the overall molecule, to provide an additional site (e.g., an amine protected by a protective group) for linking additional moieties to the compound, or to rigidize the overall molecule. A linker is attached to the remainder of the compound by any suitable linkage moiety (“linkage”), e.g., by a carbon-carbon bond, an ester, a thioester, an amine, an ether, an amide, a carbonate, or a carbamate linkage to the additional moieties of the compound. The linker may be hydrocarbyl, that is including only carbons and hydrogens (e.g. from 1 to 10 methylene groups), optionally comprising one or more hetero-atom, such as N, O, and/or S. In the context of the present invention, in one embodiment, one suitable linker is a divalent moiety comprising a PEG group —(O—CH₂—CH₂)ₙ—, where n ranges from 2 to 100, e.g., from 2-10 (PEG₂₋₁₀), or from 2-5 (PEG₂₋₅) such as 2 (PEG₂), 3, 4, 5, 6, 7, 8, 9, or 10. A PEG linker may comprise one or more methylene groups at either end in addition to a suitable linkages attaching the PEG group to connected moieties.

In another embodiment, the PNA is a gamma PNA (γPNA), which is an oligomer or polymer of gamma-modified N-(2-aminoethyl)glycine monomers where the γ carbon in formula (I), above, is a chiral center, typically with one of R₁ or R₂, attached to the gamma carbon being H and the other not a hydrogen, or R₁ and R₂ are different, such that the gamma carbon is a chiral center. When R₁ and R₂ are both hydrogen (N-(2-aminoethyl)-glycine backbone), or are the same, there is no such chirality about the gamma carbon. Alpha PNA (αPNA), is an oligomer or polymer of alpha-modified N-(2-aminoethyl)glycine monomers of the where the α carbon in formula (I), above, is a chiral center. Beta PNA (βPNA), is an oligomer or polymer of beta-modified N-(2-aminoethyl)glycine monomers of the where the β carbon in formula (I), above, is a chiral center. The discussion below regarding γPNA applies equally to αPNA and βPNA. Also, any combination of two (α,β, α,γ, or β,γ) or all three (α,β,γ) of the α, β, and γ carbons may form chiral centers.

In various embodiments, the backbone, e.g. at one or more of R₁, R₂, R₃, R₄, R₅, and R₆, is PEGylated with from 1 to 50 oxyethylene residues—that is, [—O—CH₂—CH₂—]ₙ, where n is 1 to 50, inclusive. The PEG group(s) can be linked to the backbone by any suitable linking group, such as by a C₁-6 alkyl group, or and aryl-alkyl group.

In other embodiments, the backbone, e.g., at one or more of R₁, R₂, R₃, R₄, R₅, and R₆, comprises one or more guanidine-containing groups, such as an alkyl or aryl-alkyl moiety terminated in a guanidine moiety.

In other embodiments, one or more of R₁, R₂, R₃, R₄, R₅, and R₆ are S1A, S1B, S1C, S1D, S1E, S1F, S1G, S1H, S1I, S1J, S1K, or S1L, to promote cellular uptake and endosomal escape.

An “amino acid side chain” is a side chain for an amino acid. Amino acids have the structure:

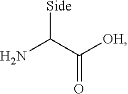

where “Side” is the amino acid side chain. Non-limiting examples of amino acid side chains include: CH₃ (Ala), CH₂OH (Ser), CH(CH₃)OH (Thr), CH₂SH (Cys), CH(CH₃)CH₃ (Val), CH₂CH(CH₃)CH₃ (Leu), CH(CH₃)CH₂CH₃ (Ile), CH₂CH₂SCH₃ (Met), 4-CH₂—C₆H₄OH (Tyr), CH₂—C₆H₅ (Phe), 1H-indol-3-yl methyl (Trp), CH₂C(O)OH (Asp), CH₂CH₂C(O)OH (Glu), CH₂C(O)NH₂ (Asn), CH₂CH₂C(O)NH₂ (Gln), 1H-imidazol-4-yl methyl (His), CH₂CH₂CH₂CH₂NH₂ (Lys), or CH₂CH₂CH₂NHC(NH)NH₂ (Arg, comprising a guanidine/guanidium group). Glycine is not represented because in the embodiment there is no side chain (Side is H).

A γPNA monomer incorporated into a γPNA oligomer or polymer is referred to herein as a “γPNA monomer residue”, with each residue having the same or different nucleobase, such as the modified nucleobases described herein, such that the order of bases on the γPNA is its “sequence”, as with DNA or RNA. A sequence of nucleobases in a nucleic acid or a nucleic acid analog oligomer or polymer, such as a γPNA oligomer or polymer, binds to a complementary sequence of adenine, guanine, cytosine, thymine and/or uracil residues in a nucleic acid strand by cooperative bonding, essentially as with Watson-Crick binding of complementary bases in double-stranded DNA or RNA. “Watson-Crick-like” bonding refers to hydrogen bonding of nucleobases other than G, A, T, C or U, such as the bonding of the divalent bases shown herein with G, A, T, C, U or other nucleobases.

Unless otherwise indicated, the nucleic acids and nucleic acid analogs described herein are not described with respect to any particular sequence of bases. The present disclosure is directed to modified nucleobases, compositions comprising the modified nucleobases, and methods of use of the modified nucleobases and compounds containing those nucleobases, and the usefulness of any specific embodiments described herein, while typically depending upon a specific sequence in each instance, is generically applicable. A nucleobase sequence attached to the backbone of γPNA oligomers can hybridize with a complementary nucleobase sequence of a target nucleic acid or nucleic acid analog by Watson-Crick or Watson-Crick-like hydrogen bonding. One of ordinary skill would understand that the compositions and methods described herein are sequence-independent and describe novel, generalized compositions comprising divalent nucleobases and related methods.

Genetic recognition reagents can be prepared as small oligonucleotides and can be assembled in situ, in vivo, ex vivo, or in vitro, for example, as described in United States Patent Application Publication No. 2016/0083433 A1, incorporated herein by reference in its entirety. By that method, small oligomers of high cell or tissue permeability as compared to longer sequences, such as trimers, can be transferred to a cell, and the oligomers can be assembled as a contiguous larger sequence once hybridized to a template nucleic acid. The same can be accomplished in vitro or ex vivo, for example, for rapidly assembling a longer sequence for use in hybridizing to a target nucleic acid.

In one aspect, the genetic recognition reagent is provided on an array. Arrays are particularly useful in implementing high-throughput assays, such as genetic detection assays. As used herein, the term “array” refers to reagents, for example the genetic recognition reagents described herein, located or attached at two or more discrete, identifiable and/or addressable locations on a substrate. In one aspect, an array is an apparatus having two or more discrete, identifiable reaction chambers, such as, without limitation a 96-well dish, in which reactions comprising identified constituents are performed. In one aspect, two or more genetic recognition reagents comprising one or more divalent nucleobases as described herein are immobilized onto a substrate in a spatially addressable manner so that each individual primer or probe is located at a different and (addressable) identifiable location on the substrate. One or more genetic recognition reagent is either covalently-linked to the substrate or are otherwise bound or located at addressable locations on the array. Substrates include, without limitation, multi-well plates, silicon chips and beads. In one aspect, the array comprises two or more sets of beads, with each bead set having an identifiable marker, such as a quantum dot or fluorescent tag, so that the beads are individually identifiable using, for example and without limitation, a flow cytometer. In one aspect, an array is a multi-well plate containing two or more wells with the described genetic recognition reagents for binding specific sequences. As such, reagents, such as probes and primers may be bound or otherwise deposited onto or into, or otherwise located at specific locations on an array. Reagents may be in any suitable form, including, without limitation: in solution, dried, lyophilized, or glassified. When linked covalently to a substrate, such as an agarose bead or silicon chip, a variety of linking technologies are known for attaching chemical moieties, such as the genetic recognition reagents to such substrates.

Linkers and spacers for use in linking nucleic acids, peptide nucleic acids and other nucleic acid analogs are broadly known in the chemical and array arts and for that reason are not described herein. As a non-limiting example, a γPNA genetic recognition reagent contains a reactive amine, which can be reacted with carboxyl-, cyanogen bromide-, N-hydroxysuccinimide ester-, carbonyldiimidazole-, or aldehyde-functional agarose beads, available, for instance from Thermo Fisher Scientific (Pierce Protein Biology Products), Rockford, Ill., and a variety of other sources. The genetic recognition reagents described herein can be attached to a substrate in any manner, with or without linkers. Devices for use in conducting reactions, and for reading arrays are broadly-known and available, and informatics and/or statistical software or other computer-implemented processes for analyzing array data and/or identifying genetic risk factors from data obtained from a patient sample, are known in the art.

Certain of the modified nucleobases described herein exhibit fluorescence, due to their ring structure. These compositions can be used as fluorochromes, or the intrinsic fluorescence can be employed as a probe, for example, by binding target sequences in an in situ assay or in a gel or blot, such that a target sequence can be visualized.

According to one aspect of the present invention, a method is provided for detection of a target sequence in a nucleic acid, comprising contacting a genetic recognition reagent composition as described herein with a sample comprising nucleic acid and detecting binding of the genetic recognition reagent with a nucleic acid. In one aspect, the genetic recognition reagent is immobilized on a substrate, for example in an array, and labeled (e.g., fluorescently labeled or radiolabeled) nucleic acid sample is contacted with the immobilized genetic recognition reagent and the amount of labeled nucleic acid specifically bound to the genetic recognition reagent is measured. In a variation, genetic recognition reagent or a nucleic acid comprising a target sequence of the genetic recognition reagent is bound to a substrate, and a labeled nucleic acid comprising a target sequence of the genetic recognition reagent or a labeled genetic recognition reagent is bound to the immobilized genetic recognition reagent or nucleic acid, respectively to form a complex. In one aspect, the nucleic acid of the complex comprises a partial target sequence so that a nucleic acid comprising the full target sequence would out-compete the complexed nucleic acid for the genetic recognition reagent. The complex is then exposed to a nucleic acid sample and loss of bound label from the complex could be detected and quantified according to standard methods, facilitating quantification of a nucleic acid marker in the nucleic acid sample. These are merely two of a large number of possible analytical assays that can be used to detect or quantify the presence of a specific nucleic acid in a nucleic acid sample.

By “immobilized” in reference to a composition such as a nucleic acid or genetic recognition reagent as described herein, it is meant attached to a substrate of any physical structure or chemical composition. The immobilized composition is immobilized by any method useful in the context of the end use. The composition is immobilized by covalent or non-covalent methods, such as by covalent linkage of amine groups to a linker or spacer, or by non-covalent bonding, including Van der Waals and/or hydrogen bonding. A “label” is a chemical moiety that is useful in detection of, or purification or a molecule or composition comprising the label. A label may be, for example and without limitation, a radioactive moiety, such as ¹⁴C, ³²P, ³⁵S, a fluorescent dye, such as fluorescein isothiocyanate or a cyanine dye, an enzyme, or a ligand for binding other compounds such as biotin for binding streptavidin, or an epitope for binding an antibody. A multitude of such labels, and methods of use thereof are known to those of ordinary skill in the immunology and molecular biology arts. That said, because certain nucleobases described herein are fluorescent, incorporation of such bases into nucleotide residues of a nucleic acid or nucleic acid analog, or covalently-linking a divalent nucleobase to a nucleic acid, nucleic acid analog, binding reagent, ligand or other detection reagent can permit detection of and/or quantification of a reagent in a sample, reaction mixture, array, etc.

In yet another aspect of the present invention, a method of isolation and purification or a nucleic acid containing a target sequence is provided. In one non-limiting aspect, a genetic recognition reagent as described herein is immobilized on a substrate, such as a bead (for example and without limitation, an agarose bead, a bead containing a fluorescent marker for sorting, or a magnetic bead), porous matrix, surface, tube, etc. A nucleic acid sample is contacted with the immobilized genetic recognition reagent and nucleic acids containing the target sequence bind to the genetic recognition reagent. The bound nucleic acid is then washed to remove unbound nucleic acids, and the bound nucleic acid is then eluted, and can be precipitated or otherwise concentrated by any useful method as are broadly known in the molecular biological arts.

In a further aspect, kits are provided. A kit comprises at a minimum a vessel of any form, including cartridges for automated nucleic acid, nucleic acid analog, or PNA synthesis, which may comprise one or more vessels in the form of individual and independent, optionally independently-addressable compartments, for use, for example, in an automatic sequence preparation device for preparing nucleic acids and/or nucleic acid analogs. Vessels may be single-use, or contain sufficient contents for multiple uses. A kit also may comprise an array. A kit may optionally comprise one or more additional reagents for use in making or using genetic recognition reagents in any embodiment described herein. The kit comprises a vessel containing any divalent nucleobase in any form described herein, or monomers or genetic recognition reagents according to any aspect described herein. Different nucleobases, monomers or genetic recognition reagents are typically packaged into separate vessels, which may be separate compartments in a cartridge.

In aspects, the compounds and genetic recognition reagents are used for therapeutic purposes and therefore those compounds and genetic recognition reagents are formulated in a drug product, pharmaceutical composition, or dosage form, including compositions for human and veterinary use, including a therapeutically effective amount of the compound or genetic recognition reagent and an excipient, e.g., a vehicle or diluent for therapeutic delivery, e.g., and without limitation, for oral, topical, intravenous, intramuscular, or subcutaneous administration. The composition can be formulated in a classical manner using solid or liquid vehicles, diluents and additives appropriate to the desired mode of administration. Orally, the compounds can be administered in the form of tablets, capsules, granules, powders and the like. The compositions optionally comprise one or more additional active agents, as are broadly known in the pharmaceutical, medicinal, veterinary or biological arts. The compounds described herein may be administered in any effective manner. Further examples of delivery routes include, without limitation: topical, for example, epicutaneous, inhalational, enema, ocular, otic and intranasal delivery; enteral, for example, orally, by gastric feeding tube or swallowing, and rectally; and parenteral, such as, intravenous, intraarterial, intramuscular, intracardiac, subcutaneous, intraosseous, intradermal, intrathecal, intraperitoneal, transdermal, iontophoretic, transmucosal, epidural and intravitreal. Therapeutic/pharmaceutical compositions are prepared in accordance with acceptable pharmaceutical procedures, as are broadly-known.

Any of the compounds described herein may be compounded or otherwise manufactured into a suitable composition for use, such as a pharmaceutical dosage form or drug product in which the compound or genetic recognition reagent is an active ingredient. According to one example, the drug product described herein is an oral tablet, capsule, caplet, liquid-filled or gel-filled capsule, etc. Compositions may comprise a pharmaceutically acceptable carrier, or excipient. An “excipient” is an inactive substance used as a carrier for the active ingredients of a medication. Although “inactive,” excipients may facilitate and aid in increasing the delivery, stability or bioavailability of an active ingredient in a drug product. Non-limiting examples of useful excipients include: antiadherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts.

### Example—Synthesis of Modified Nucleobases and Cell-Permeable γPNAs

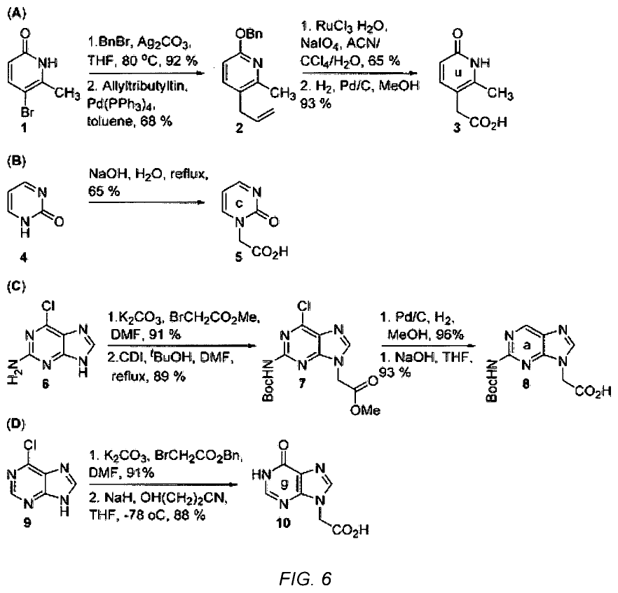

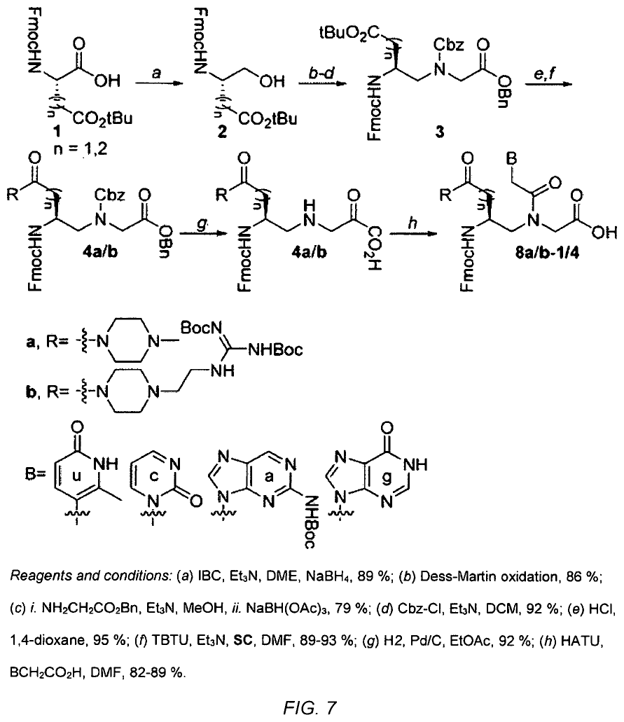

The compounds and genetic recognition reagents described herein are synthesized according to methods known in the chemical and organic synthesis arts. Illustrative synthesis schemes are provided in FIGS. 6 and 7.

The following numbered clauses describe non-limiting embodiments and aspects of the invention.

Clause 1: A genetic recognition reagent comprising a plurality of nucleobase moieties attached to a nucleic acid or nucleic acid analog backbone, in which at least one nucleobase moiety is:

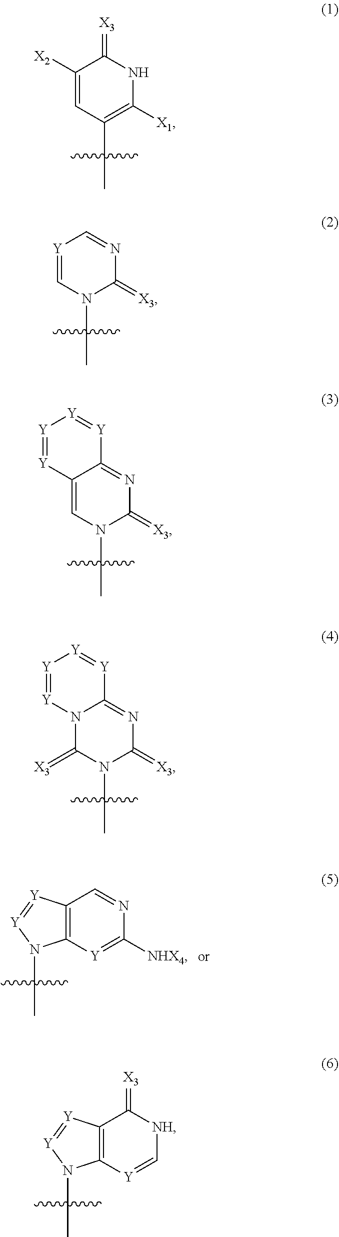

wherein,
X₁ is ═O, ═S, ═Se, or CH₃;
X₂ is H, CH₃, CN, NC, N₃, C(O)OH, or C(O)NH₂;

### X₃ is O or S;

X₄ is H, C(O)CH₃, or C(O)OCH₃; and

### Y is N or CH,

wherein in (1), when X₁ and X₃ are O, X₂ is not H or methyl.
Clause 2: The genetic recognition reagent of clause 1, wherein the at least one nucleobase moiety is:

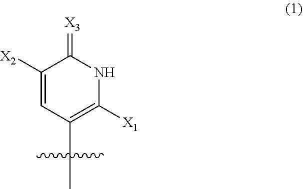

Clause 3: The genetic recognition reagent of clause 1, wherein the at least one nucleobase moiety is:

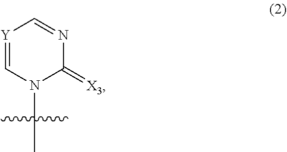

Clause 4: The genetic recognition reagent of clause 1, wherein the at least one nucleobase moiety is:

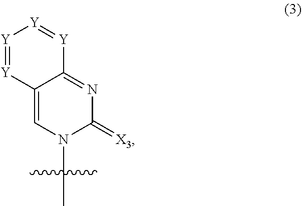

Clause 5: The genetic recognition reagent of clause 1, wherein the at least one nucleobase moiety is:

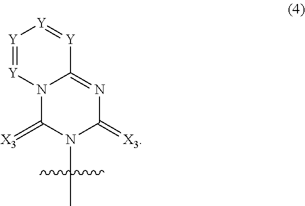

Clause 6: The genetic recognition reagent of clause 1, wherein the at least one nucleobase moiety is:

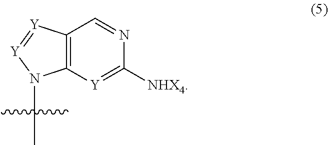

Clause 7: The genetic recognition reagent of clause 1, wherein the at least one nucleobase moiety is:

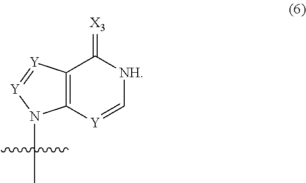

Clause 8: The genetic recognition reagent of clause 1, wherein the at least one nucleobase moiety is:

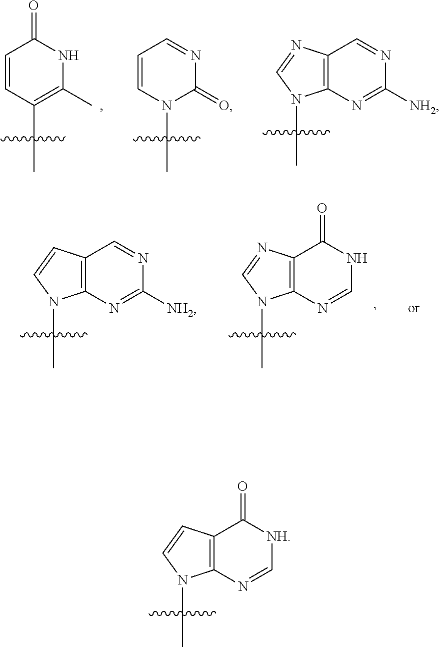

Clause 9: The genetic recognition reagent of any one of clauses 1-8, in which the backbone is chosen from one of a DNA, RNA, peptide nucleic acid (PNA), phosphorothioate DNA (PS DNA), α, β-constrained nucleic acid (α,β-CNA), 2′-methoxyl RNA, 2′-fluoro RNA, locked nucleic acid (LNA), 2′,4′-constrained ethyl nucleic acid ((S)-cEt), 2′,4′-bridged nucleic acid NC (N—H) (BNA-NC(N—H)), 2′,4′ bridged nucleic acid NC (N-methyl) (BNA-NC(N-Me)), 2′-(R)-(S)-5′-C-methyl DNA, or 2′-R-5′-E-vinylphosphonate nucleic acid (E-VP), wherein R is H, OH, F, OMe, or O(CH₂)₂OMe backbone.
Clause 10: The genetic recognition reagent of clause 1, in which the backbone is a peptide nucleic acid (PNA) backbone.
Clause 11: The genetic recognition reagent of clause 10, wherein the backbone is PEGylated, with one or more PEG moieties of two to fifty (—O—CH₂—CH₂—) residues linked to the backbone.
Clause 12: The genetic recognition reagent of clause 10, wherein the backbone comprises one or more guanidine moieties linked to the backbone.
Clause 13: The genetic recognition reagent of clause 1, in which the backbone is a gamma peptide nucleic acid (γPNA) backbone.
Clause 14: The genetic recognition reagent of clause 1, in which the backbone is a PNA backbone comprising the residue

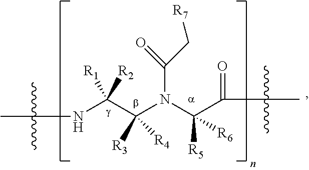

where n is 1 or greater, R₁, R₂, R₃, R₄, R₅, and R₆ are, independently: H; CH₃, CH₂OH, CH(CH₃)OH, CH₂SH, CH(CH₃)CH₃, CH₂CH(CH₃)CH₃, CH(CH₃)CH₂CH₃, CH₂CH₂SCH₃, CH₂CH₃, CH₂—C₆H₅, CH₂—C₆H₄OH, 1H-indol-3-yl methyl, CH₂C(O)OH, CH₂CH₂C(O)OH, CH₂C(O)N H₂, CH₂CH₂C(O)N H₂, 1 H-imidazol-4-yl methyl, CH₂CH₂CH₂CH₂NH₂, or CH₂CH₂CH₂NHC(NH)NH₂; linear or branched (C₃-C₈)alkyl, (C₂-C₈)alkenyl, (C₂-C₈)alkynyl, (C₃-C₈)aryl, (C₃-C₈)cycloalkyl, (C₃-C₈)aryl(C₁-C₆)alkylene, (C₃-C₈)cycloalkyl(C₁-C₆)alkylene, a guanidine-containing group, CH₂—(OCH₂—CH₂)ₙ—OH, CH₂—(OCH₂—CH₂)ₙ—NH₂, CH₂—(OCH₂—CH₂)ₙ—SH, CH₂—(OCH₂—CH₂)ₙ—NHC(NH)NH₂, CH₂—(OCH₂—CH₂)ₙ-morpholine, CH₂—(OCH₂—CH₂)ₙ-piperazine,

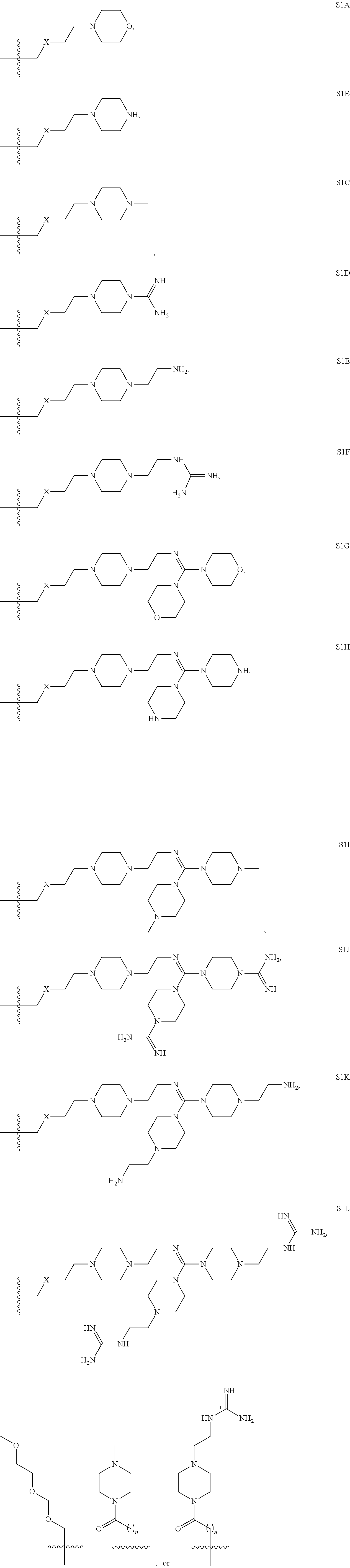

wherein X is a linker, R₁ and R₂ together form a 1,3-propylene linkage, R₃ and R₄ together form a 1,3-propylene linkage, or R₅ and R₆ together form a 1,3-propylene linkage, and each instance of R₇ is, independently, a nucleobase.

Clause 15: The genetic recognition reagent of clause 14, wherein at least one of R₁, R₂, R₃, R₄, R₅, and R₆ is S1A, S1B, S1C, S1D, S1E, S1F, S1G, S1H, S1I, S1J, S1K, or S1L.

Clause 16: The genetic recognition reagent of clause 14 or 15, wherein the α-carbon, the β-carbon, or the γ-carbon is a chiral center.
Clause 17: The genetic recognition reagent of clause 16, wherein the γ-carbon is a chiral center.
Clause 18: The genetic recognition reagent of clause 17, wherein R₂ is:

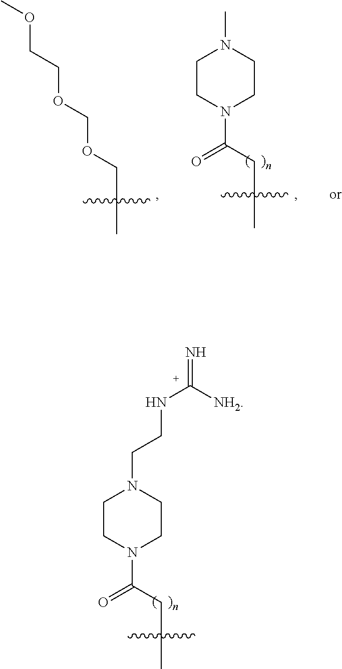

Clause 19: The genetic recognition reagent of clause 16, wherein R₁, R₃, R₄, R₅, and R₆ are H.
Clause 20: The genetic recognition reagent of any one of clauses 1-19, comprising end-groups linked to the nucleic acid or nucleic acid analog backbone, for self-assembly of two or more adjacent genetic recognition reagents on a nucleic acid template.
Clause 21: The genetic recognition reagent of clause 20, wherein the end groups are two- to five-ring fused polycyclic aromatic moieties.
Clause 22: The genetic recognition reagent of clause 20, wherein the end groups are sulfhydryl or thioester groups.
Clause 23: The genetic recognition reagent of any one of clauses 1-22, wherein the plurality of nucleobase moieties form a sequence that comprises: TTC, TTCTTC, TCT, TCTTCT, CTT, CTTCTT, CCG, CCGCCG, CGC, CGCCGC, GCC, GCCGCC, CGG, CGGCGG, GCG, GCGGCG, GGC, GGCGGC, CTG, CTGCTG, TGC, TGCTGC, GCT, GCTGCT, CAG, CAGCAG, AGC, AGCAGC, GCA, GCAGCA, CAGG, CAGGCAGG, AGGC, AGGCAGGC, GGCA, GGCAGGCA, GCAG, GCAGGCAG, AGAAT, GAATA, AATAG, ATAGA, TAGAA, GGCCCC, GCCCCG, CCCCGG, CCCGGC, CCGGCC, and CGGCCC, or a contiguous repeat of any of the preceding.
Clause 24: The genetic recognition reagent of any one of clauses 1-23, in which the plurality of nucleobase moieties is arranged in a sequence complementary to a target sequence of a nucleic acid.
Clause 25: The genetic recognition reagent of any one of clauses 1-24, having from 3 to 25 nucleobase moieties.
Clause 26: A compound comprising a nucleic acid backbone monomer or nucleic acid analog backbone monomer linked to a nucleobase moiety having the structure:

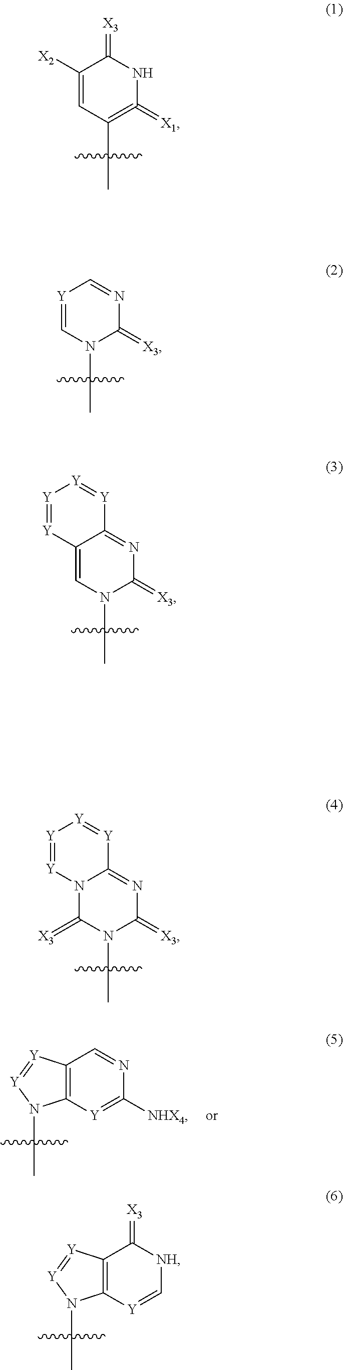

wherein,
X₁ is ═O, ═S, ═Se, or CH₃;
X₂ is H, CH₃, CN, NC, N₃, C(O)OH, or C(O)NH₂;

### X₃ is O or S;

X₄ is H, C(O)CH₃, or C(O)OCH₃; and

### Y is N or CH,

wherein in (I), when X₁ and X₃ are O, X₂ is not H or methyl.
Clause 27: The compound of clause 26, wherein the at least one nucleobase moiety is:

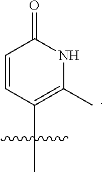 [SMILES: *c1ccc(=O)[nH]c1C]

Clause 28: The compound of clause 26, wherein the at least one nucleobase moiety is:

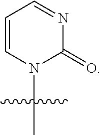 [SMILES: *n1cccnc1=O]

Clause 29: The compound of clause 26, wherein the at least one nucleobase moiety is:

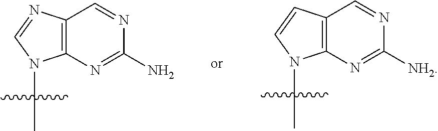 [SMILES: *n1cnc2cnc(N)nc21 ; *n1ccc2cnc(N)nc21]

Clause 30: The compound of clause 26, wherein the at least one nucleobase moiety is:

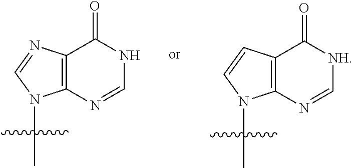 [SMILES: *n1cnc2c(=O)[nH]cnc21 ; *n1ccc2c(=O)[nH]cnc21]

Clause 31: The compound of any one of clauses 26-30, in which the backbone monomer is a DNA, RNA, peptide nucleic acid (PNA), phosphorothioate DNA (PS DNA), α, β-constrained nucleic acid (α,β-CNA), 2′-methoxyl RNA, 2′-fluoro RNA, locked nucleic acid (LNA), 2′,4′-constrained ethyl nucleic acid ((S)-cEt), 2′,4′-bridged nucleic acid NC (N—H) (BNA-NC(N—H)), 2′,4′ bridged nucleic acid NC (N-methyl) (BNA-NC(N-Me)), 2′-(R)-(S)-5′-C-methyl DNA, or 2′-R-5′-E-vinylphosphonate nucleic acid (E-VP), wherein R is H, OH, F, OMe, or O(CH₂)₂OMe backbone monomer.
Clause 32: The compound of any one of clauses 26-31, in which the backbone monomer is a peptide nucleic acid (PNA) backbone monomer.
Clause 33: The compound of clause 32, wherein the backbone monomer is PEGylated, with one or more PEG moieties of two to fifty (—O—CH₂—CH₂—) residues linked to the backbone monomer.
Clause 34: The compound of clause 32, wherein the backbone comprises one or more guanidine moieties linked to the backbone.
Clause 35: The compound of clause 26, in which the backbone monomer is a gamma peptide nucleic acid (γPNA) backbone monomer.
Clause 36: The compound of clause 26, in which the backbone monomer is a PNA backbone having the structure

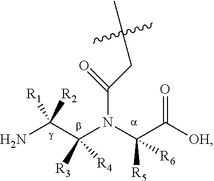

where R₁, R₂, R₃, R₄, R₅, and R₆ are, independently: H; CH₃, CH₂OH, CH(CH₃)OH, CH₂SH, CH(CH₃)CH₃, CH₂CH(CH₃)CH₃, CH(CH₃)CH₂CH₃, CH₂CH₂SCH₃, CH₂CH₃, CH₂—C₆H₅, 1 H-indol-3-yl methyl, CH₂—C₆H₄OH, CH₂C(O)OH, CH₂CH₂C(O)OH, CH₂C(O)NH₂, CH₂CH₂C(O)NH₂, 1 H-imidazol-4-yl methyl, CH₂CH₂CH₂CH₂NH₂, or CH₂CH₂CH₂NHC(NH)NH₂; linear or branched (C₃-C₈)alkyl, (C₂-C₈)alkenyl, (C₂-C₈)alkynyl, (C₃-C₈)aryl, (C₃-C₈)cycloalkyl, (C₃-C₈)aryl(C₁-C₆)alkylene, (C₃-C₈)cycloalkyl(C₁-C₆)alkylene, a guanidine-containing group, CH₂—(OCH₂—CH₂)ₙ—OH, CH₂—(OCH₂—CH₂)ₙ—NH₂, CH₂—(OCH₂—CH₂)ₙ—SH, CH₂—(OCH₂—CH₂)ₙ—NHC(NH)NH₂, CH₂—(OCH₂—CH₂)ₙ-morpholine, CH₂—(OCH₂—CH₂)ₙ-piperazine,

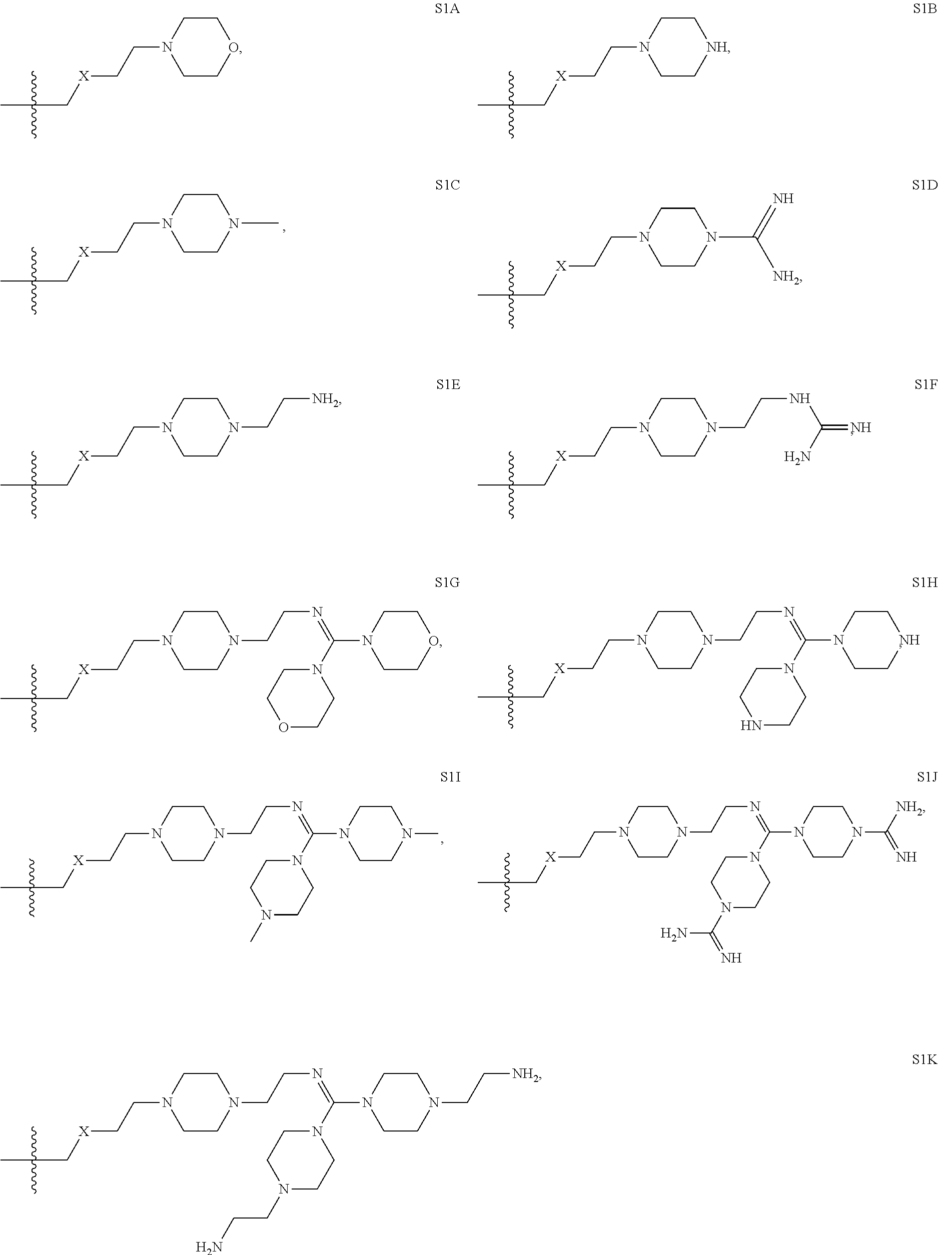

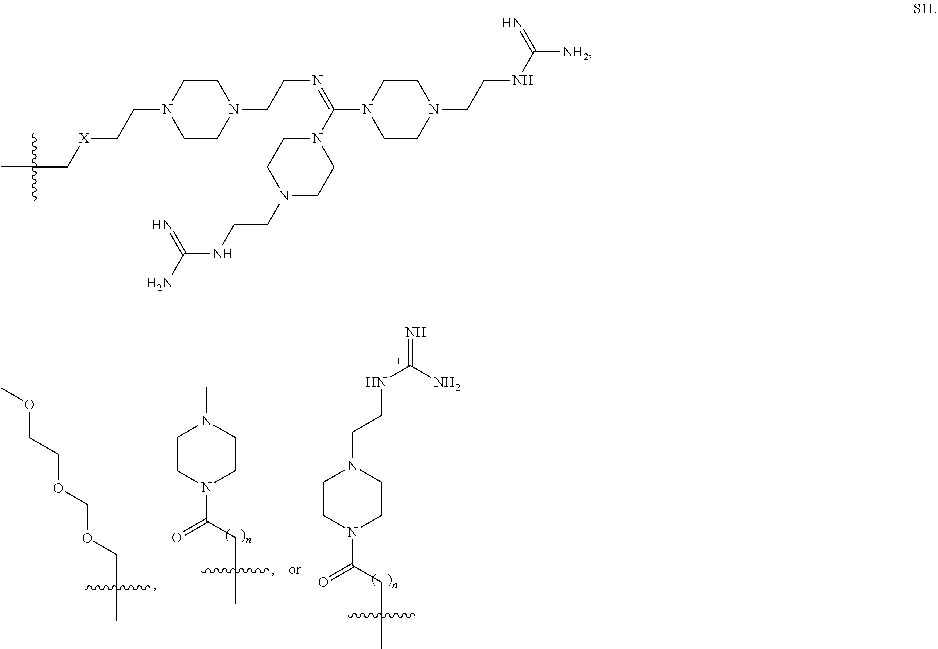

wherein X is a linker, R₁ and R₂ together form a 1,3-propylene linkage, R₃ and R₄ together form a 1,3-propylene linkage, or R₅ and R₆ together form a 1,3-propylene linkage, and each instance of R7 is, independently, a nucleobase.
Clause 37: The compound of clause 36, wherein at least one of R₁, R₂, R₃, R₄, R₅, and R₆ is S1A, S1B, S1C, S1D, S1E, S1F, S1G, S1H, S1I, S1J, S1K, or S1L.
Clause 38: The compound of clause 36 or 37, wherein the α-carbon, the β-carbon, or the γ-carbon is a chiral center.
Clause 39: The compound of clause 38, wherein the γ-carbon is a chiral center.
Clause 40: The compound of clause 39, wherein R₂ is:

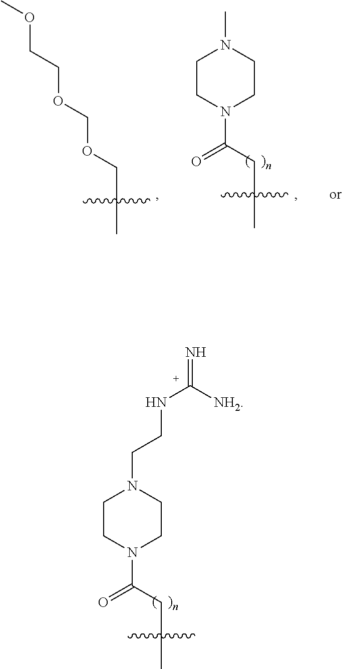

Clause 41: The compound of clause 37, wherein R₁, R₃, R₄, R₅, and R₆ are H
Clause 42: A kit comprising a compound of any one of clauses 36-41 in a vessel.
Clause 43: The kit of clause 42, comprising, in separate vessels, monomers binding adenine, guanine, cytosine and either or both of uracil and thymine.
Clause 44: A kit comprising a genetic recognition reagent of any one of clauses 1-25 in a vessel.
Clause 45: The kit of any one of clauses 42-44, wherein the vessel(s) is or are compartment(s) in a cartridge for use in an automated device.
Clause 46: An array comprising a genetic recognition reagent of any one of clauses 1-19.
Clause 47: A method of detection of a target sequence in a nucleic acid, comprising contacting a genetic recognition reagent of any one of clauses 1-25 with a sample comprising nucleic acid and detecting binding of the genetic recognition reagent with a nucleic acid.
Clause 48: A method of isolation and purification or a nucleic acid containing a target sequence, comprising, contacting a nucleic acid sample with a genetic recognition reagent of any of clauses 1-29, separating the nucleic acid sample from the genetic recognition reagent, leaving any nucleic acid bound to the genetic recognition reagent bound to the genetic recognition reagent, and separating the genetic recognition reagent from any nucleic acid bound to the genetic recognition reagent.
Clause 49: The method of clause 48, wherein the genetic recognition reagent is immobilized on a substrate, comprising contacting a nucleic acid with the substrate, washing the substrate to remove unbound nucleic acid from the substrate, but leaving bound nucleic acid bound to the substrate, and eluting the bound nucleic acid from the substrate.
Clause 50: A composition comprising a genetic recognition reagent or compound according to any one of clauses 1-49, and a pharmaceutically-acceptable excipient.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

We claim:

1. A genetic recognition reagent comprising a plurality of nucleobase moieties attached to a nucleic acid or nucleic acid analog backbone, in which at least one nucleobase moiety is: 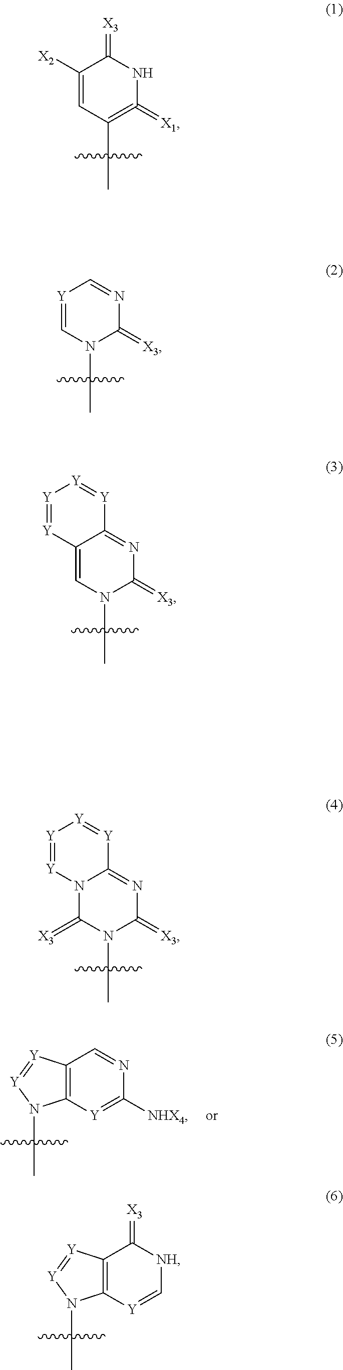
wherein,
X₁ is ═O, ═S, ═Se, or CH₃;
X₂ is H, CH₃, CN, NC, N₃, C(O)OH, or C(O)NH₂;
X₃ is O or S;
X₄ is H, C(O)CH₃, or C(O)OCH₃; and
Y is N or CH,
wherein in (1), when X₁ and X₃ are O, X₂ is not H or methyl.

2. The genetic recognition reagent of claim 1, wherein the at least one nucleobase moiety is:

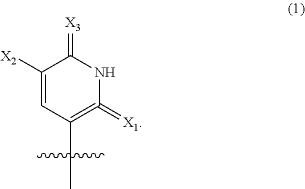

3. The genetic recognition reagent of claim 1, wherein the at least one nucleobase moiety is:

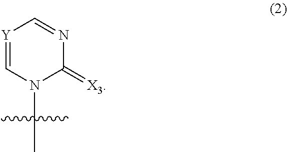

4. The genetic recognition reagent of claim 1, wherein the at least one nucleobase moiety is:

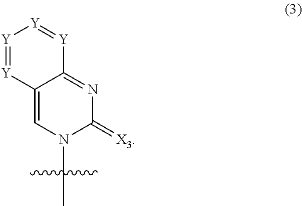

5. The genetic recognition reagent of claim 1, wherein the at least one nucleobase moiety is:

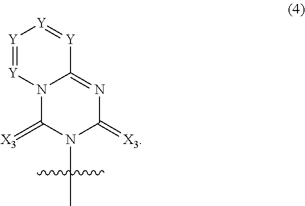

6. The genetic recognition reagent of claim 1, wherein the at least one nucleobase moiety is:

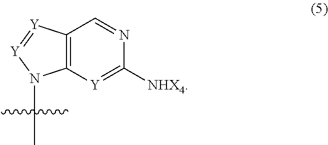

7. The genetic recognition reagent of claim 1, wherein the at least one nucleobase moiety is:

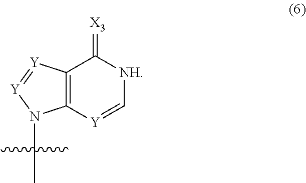

8. The genetic recognition reagent of claim 1, wherein the at least one nucleobase moiety is:

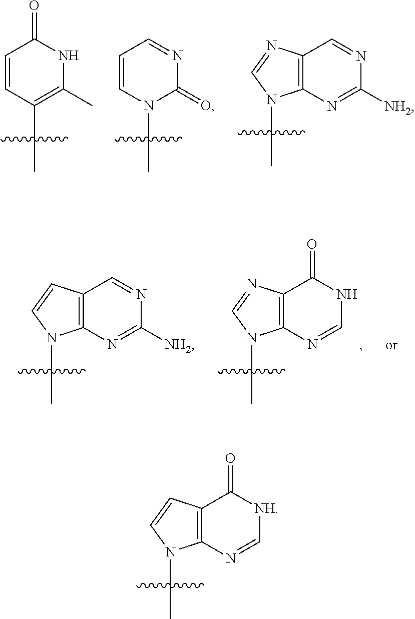

9. The genetic recognition reagent of any one of claims 1-8, in which the backbone is chosen from one of a DNA, RNA, peptide nucleic acid (PNA), phosphorothioate DNA (PS DNA), α, β-constrained nucleic acid (α,β-CNA), 2′-methoxyl RNA, 2′-fluoro RNA, locked nucleic acid (LNA), 2′,4′-constrained ethyl nucleic acid ((S)-cEt), 2′,4′-bridged nucleic acid NC (N—H) (BNA-NC(N—H)), 2′,4′ bridged nucleic acid NC (N-methyl) (BNA-NC(N-Me)), 2′-(R)-(S)-5′-C-methyl DNA, or 2′-R-5′-E-vinylphosphonate nucleic acid (E-VP), wherein R is H, OH, F, OMe, or O(CH₂)₂OMe backbone.

10. The genetic recognition reagent of claim 1, in which the backbone is a peptide nucleic acid (PNA) backbone.

11. The genetic recognition reagent of claim 10, wherein the backbone is PEGylated, with one or more PEG moieties of two to fifty (—O—CH₂—CH₂—) residues linked to the backbone.

12. The genetic recognition reagent of claim 10, wherein the backbone comprises one or more guanidine moieties linked to the backbone.

13. The genetic recognition reagent of claim 1, in which the backbone is a gamma peptide nucleic acid (γPNA) backbone.

14. The genetic recognition reagent of claim 1, in which the backbone is a PNA backbone comprising the residue 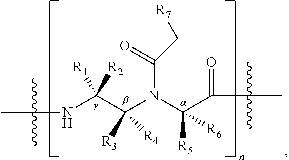
where n is 1 or greater, R₁, R₂, R₃, R₄, R₅, and R₆ are, independently: H; CH₃, CH₂OH, CH(CH₃)OH, CH₂SH, CH(CH₃)CH₃, CH₂CH(CH₃)CH₃, CH(CH₃)CH₂CH₃, CH₂CH₂SCH₃, CH₂CH₃, CH₂—C₆H₅, CH₂—C₆H₄OH, 1H-indol-3-yl methyl, CH₂C(O)OH, CH₂CH₂C(O)OH, CH₂C(O)NH₂, CH₂CH₂C(O)NH₂, 1H-imidazol-4-yl methyl, CH₂CH₂CH₂CH₂NH₂, or CH₂CH₂CH₂NHC(NH)NH₂; linear or branched (C₃-C₈)alkyl, (C₂-C₈)alkenyl, (C₂-C₈)alkynyl, (C₃-C₈)aryl, (C₃-C₈)cycloalkyl, (C₃-C₈)aryl(C₁-C₆)alkylene, (C₃-C₈)cycloalkyl(C₁-C₆)alkylene, a guanidine-containing group, CH₂—(OCH₂—CH₂)ₙ—OH, CH₂—(OCH₂—CH₂)ₙ—NH₂, CH₂—(OCH₂—CH₂)ₙ—SH, CH₂—(OCH₂—CH₂)ₙ—NHC(NH)NH₂, CH₂—(OCH₂—CH₂)ₙ-morpholine, CH₂—(OCH₂—CH₂)ₙ-piperazine, 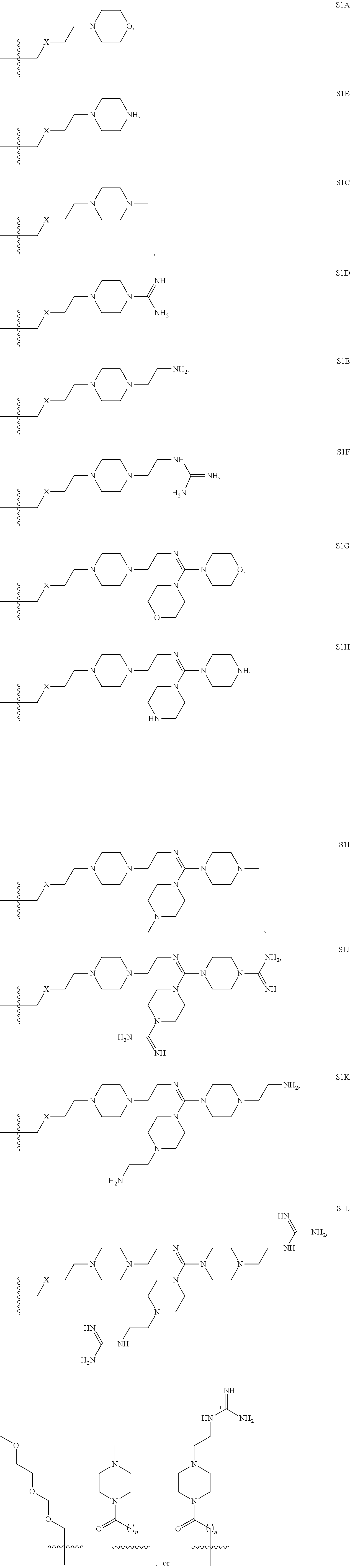
wherein X is a linker, R₁ and R₂ together form a 1,3-propylene linkage, R₃ and R₄ together form a 1,3-propylene linkage, or R₅ and R₆ together form a 1,3-propylene linkage, and each instance of R₇ is, independently, a nucleobase.

15. The genetic recognition reagent of claim 14, wherein at least one of R₁, R₂, R₃, R₄, R₅, and R₅ is S1A, S1B, S1C, S1D, S1E, S1F, S1G, S1H, S1I, S1J, S1K, or S1L.

16. The genetic recognition reagent of claim 14 or 15, wherein the α-carbon, the β-carbon, or the γ-carbon is a chiral center.

17. The genetic recognition reagent of claim 16, wherein the γ-carbon is a chiral center.

18. The genetic recognition reagent of claim 17, wherein R₂ is:

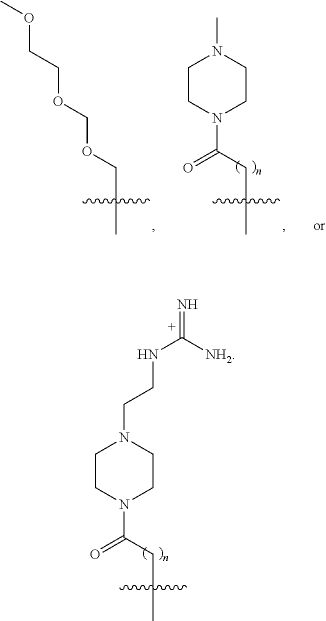

19. The genetic recognition reagent of claim 16, wherein R₁, R₃, R₄, R₅, and R₆ are H.

20. The genetic recognition reagent of any one of claims 1-19, comprising end-groups linked to the nucleic acid or nucleic acid analog backbone, for self-assembly of two or more adjacent genetic recognition reagents on a nucleic acid template.

21. The genetic recognition reagent of claim 20, wherein the end groups are two- to five-ring fused polycyclic aromatic moieties.

22. The genetic recognition reagent of claim 20, wherein the end groups are sulfhydryl or thioester groups.

23. The genetic recognition reagent of any one of claims 1-22, wherein the plurality of nucleobase moieties form a sequence that comprises: TTC, TTCTTC, TCT, TCTTCT, CTT, CTTCTT, CCG, CCGCCG, CGC, CGCCGC, GCC, GCCGCC, CGG, CGGCGG, GCG, GCGGCG, GGC, GGCGGC, CTG, CTGCTG, TGC, TGCTGC, GCT, GCTGCT, CAG, CAGCAG, AGC, AGCAGC, GCA, GCAGCA, CAGG, CAGGCAGG, AGGC, AGGCAGGC, GGCA, GGCAGGCA, GCAG, GCAGGCAG, AGAAT, GAATA, AATAG, ATAGA, TAGAA, GGCCCC, GCCCCG, CCCCGG, CCCGGC, CCGGCC, and CGGCCC, or a contiguous repeat of any of the preceding.

24. The genetic recognition reagent of any one of claims 1-23, in which the plurality of nucleobase moieties is arranged in a sequence complementary to a target sequence of a nucleic acid.

25. The genetic recognition reagent of any one of claims 1-24, having from 3 to 25 nucleobase moieties.

26. A compound comprising a nucleic acid backbone monomer or nucleic acid analog backbone monomer linked to a nucleobase moiety having the structure: 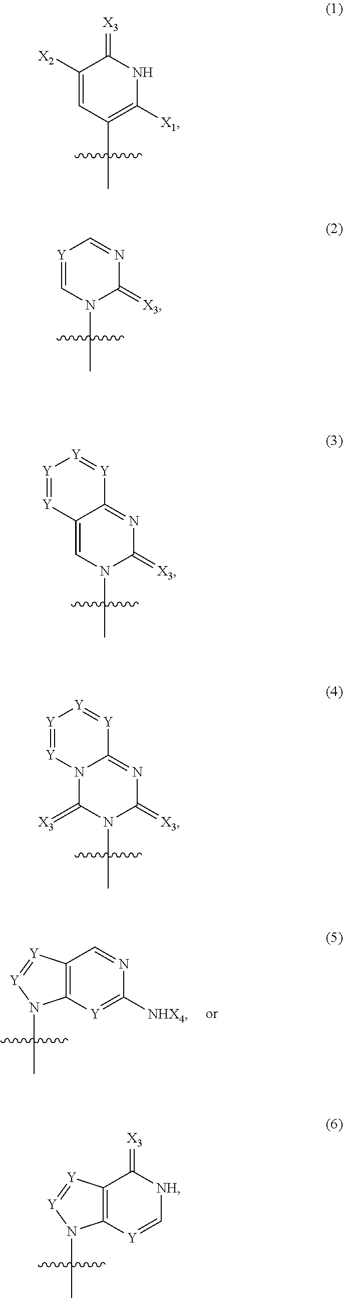
wherein,
X₁ is ═O, ═S, ═Se, or CH₃;
X₂ is H, CH₃, CN, NC, N₃, C(O)OH, or C(O)NH₂,
X₃ is O or S;
X₄ is H, C(O)CH₃, or C(O)OCH₃; and
Y is N or CH,
wherein in (I), when X₁ and X₃ are O, X₂ is not H or methyl.

27. The compound of claim 26, wherein the at least one nucleobase moiety is:

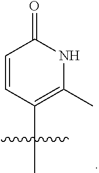 [SMILES: *c1ccc(=O)[nH]c1C]

28. The compound of claim 26, wherein the at least one nucleobase moiety is:

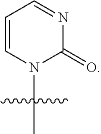 [SMILES: *n1cccnc1=O]

29. The compound of claim 26, wherein the at least one nucleobase moiety is:

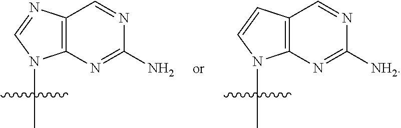 [SMILES: *n1cnc2cnc(N)nc21 ; *n1ccc2cnc(N)nc21]

30. The compound of claim 26, wherein the at least one nucleobase moiety is:

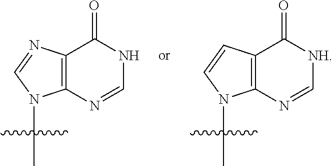 [SMILES: *n1cnc2c(=O)[nH]cnc21 ; "or" ; *n1ccc2c(=O)[nH]cnc21]

31. The compound of any one of claims 26-30, in which the backbone monomer is a DNA, RNA, peptide nucleic acid (PNA), phosphorothioate DNA (PS DNA), α, β-constrained nucleic acid (α,β-CNA), 2′-methoxyl RNA, 2′-fluoro RNA, locked nucleic acid (LNA), 2′,4′-constrained ethyl nucleic acid ((S)-cEt), 2′,4′-bridged nucleic acid NC (N—H) (BNA-NC(N—H)), 2′,4′ bridged nucleic acid NC (N-methyl) (BNA-NC(N-Me)), 2′-(R)-(S)-5′-C-methyl DNA, or 2′-R-5′-E-vinylphosphonate nucleic acid (E-VP), wherein R is H, OH, F, OMe, or O(CH₂)₂OMe backbone monomer.

32. The compound of any one of claims 26-31, in which the backbone monomer is a peptide nucleic acid (PNA) backbone monomer.

33. The compound of claim 32, wherein the backbone monomer is PEGylated, with one or more PEG moieties of two to fifty (—O—CH₂—CH₂—) residues linked to the backbone monomer.

34. The compound of claim 32, wherein the backbone comprises one or more guanidine moieties linked to the backbone.

35. The compound of claim 26, in which the backbone monomer is a gamma peptide nucleic acid (γPNA) backbone monomer.

36. The compound of claim 26, in which the backbone monomer is a PNA backbone having the structure 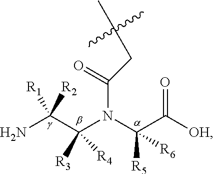
where R₁, R₂, R₃, R₄, R₅, and R₆ are, independently: H; CH₃, CH₂OH, CH(CH₃)OH, CH₂SH, CH(CH₃)CH₃, CH₂CH(CH₃)CH₃, CH(CH₃)CH₂CH₃, CH₂CH₂SCH₃, CH₂CH₃, CH₂—C₆H₅, 1H-indol-3-yl methyl, CH₂—C₆H₄OH, CH₂C(O)OH, CH₂CH₂C(O)OH, CH₂C(O)NH₂, CH₂CH₂C(O)NH₂, 1H-imidazol-4-yl methyl, CH₂CH₂CH₂CH₂NH₂, or CH₂CH₂CH₂NHC(NH)NH₂; linear or branched (C₃-C₈)alkyl, (C₂-C₈)alkenyl, (C₂-C₈)alkynyl, (C₃-C₈)aryl, (C₃-C₈)cycloalkyl, (C₃-C₈)aryl(C₁-C₆)alkylene, (C₃-C₈)cycloalkyl(C₁-C₆)alkylene, a guanidine-containing group, CH₂—(OCH₂—CH₂)ₙ—OH, CH₂—(OCH₂—CH₂)ₙ—NH₂, CH₂—(OCH₂—CH₂)ₙ—SH, CH₂—(OCH₂—CH₂)ₙ—NHC(NH)NH₂, CH₂—(OCH₂—CH₂)ₙ-morpholine, CH₂—(OCH₂—CH₂)ₙ-piperazine, 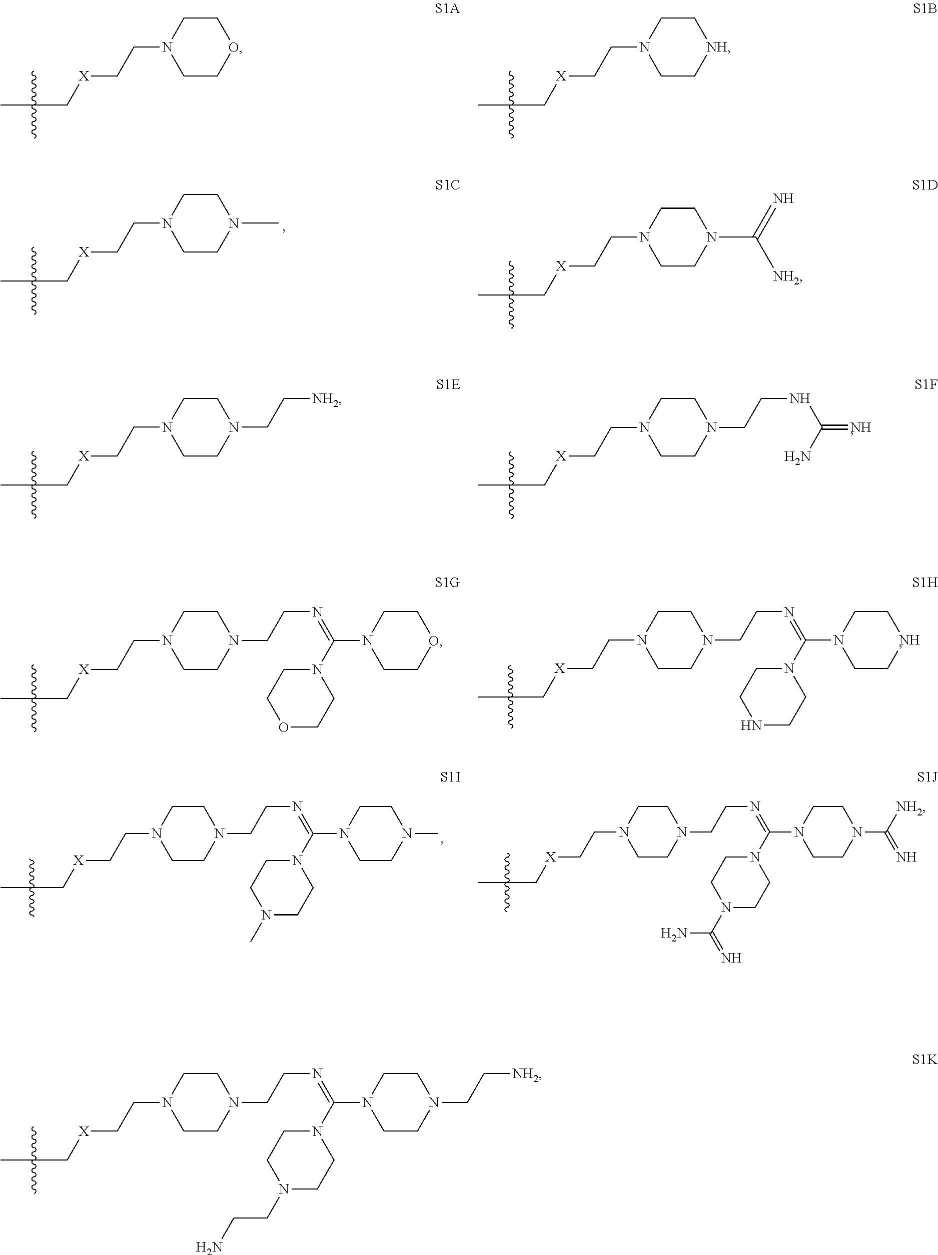 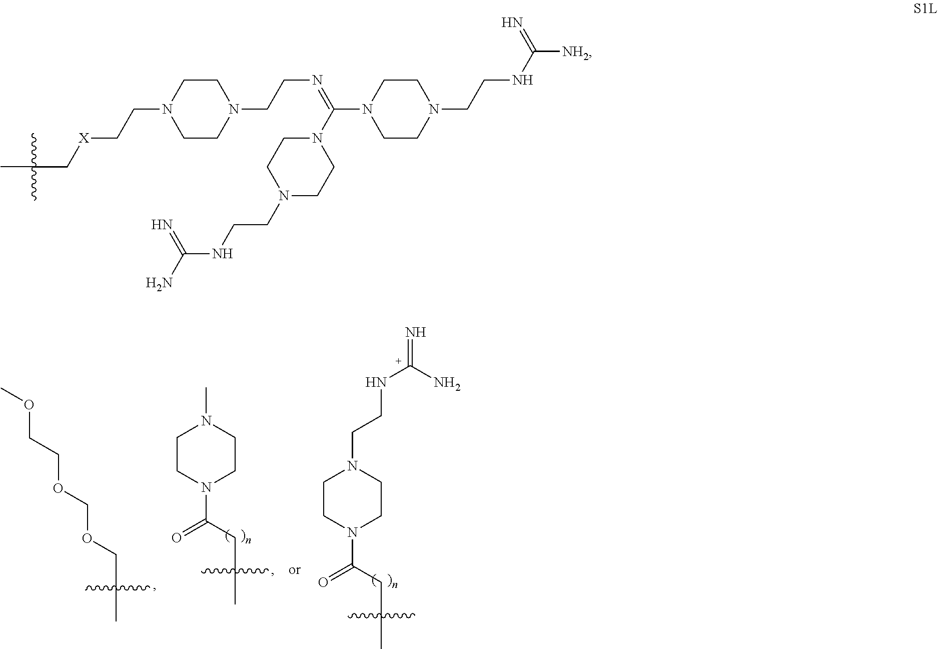
wherein X is a linker, R₁ and R₂ together form a 1,3-propylene linkage, R₃ and R₄ together form a 1,3-propylene linkage, or R₅ and R₆ together form a 1,3-propylene linkage, and each instance of R₇ is, independently, a nucleobase.

37. The compound of claim 36, wherein at least one of R₁, R₂, R₃, R₄, Rb, and R₆ is S1A, S1B, S1C, S1D, S1E, S1F, S1G, S1H, S1I, S1J, S1K, or S1L.

38. The compound of claim 36 or 37, wherein the α-carbon, the β-carbon, or the γ-carbon is a chiral center.

39. The compound of claim 38, wherein the γ-carbon is a chiral center.

40. The compound of claim 39, wherein R₂ is:

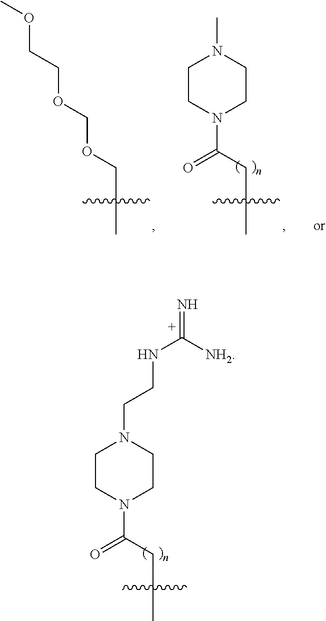

41. The compound of claim 37, wherein R₁, R₃, R₄, R₅, and R₆ are H.

42. A kit comprising a compound of any one of claims 36-41 in a vessel.

43. The kit of claim 42, comprising, in separate vessels, monomers binding adenine, guanine, cytosine and either or both of uracil and thymine.

44. A kit comprising a genetic recognition reagent of any one of claims 1-25 in a vessel.

45. The kit of any one of claims 42-44, wherein the vessel(s) is or are compartment(s) in a cartridge for use in an automated device.

46. An array comprising a genetic recognition reagent of any one of claims 1-19.

47. A method of detection of a target sequence in a nucleic acid, comprising contacting a genetic recognition reagent of any one of claims 1-25 with a sample comprising nucleic acid and detecting binding of the genetic recognition reagent with a nucleic acid.

48. A method of isolation and purification or a nucleic acid containing a target sequence, comprising, contacting a nucleic acid sample with a genetic recognition reagent of any of claims 1-29, separating the nucleic acid sample from the genetic recognition reagent, leaving any nucleic acid bound to the genetic recognition reagent bound to the genetic recognition reagent, and separating the genetic recognition reagent from any nucleic acid bound to the genetic recognition reagent.

49. The method of claim 48, wherein the genetic recognition reagent is immobilized on a substrate, comprising contacting a nucleic acid with the substrate, washing the substrate to remove unbound nucleic acid from the substrate, but leaving bound nucleic acid bound to the substrate, and eluting the bound nucleic acid from the substrate.

50. A composition comprising a genetic recognition reagent or compound according to any one of claims 1-49, and a pharmaceutically-acceptable excipient.
